(19) United States
(12) Patent Application Publication
(10) Pub. No.: US 2003/0129594 A1
(43) Pub. Date: Jul. 10, 2003

(54) Genes and the physics of the DNA double-helix. formulation of a physics-based gene identification (PBGI) method: ab initio identification of genes in eukaryotic genomes

(76) Inventor: Edouard Yeramian, Paris (FR)

Correspondence Address: FINNEGAN, HENDERSON, FARABOW,, GARRETT & DUNNER, L.L.P., 1300 I STREET, N.W., WASHINGTON, DC, 20005-3315, US

(21) Appl. No.: 09/950,051

(22) Filed: Sep. 12, 2001

Related U.S. Application Data
(60) Provisional application No. 60/232,146, filed on Sep. 13, 2000.

(51) Int. Cl. C12Q 1/68; G06F 19/00; G01N 33/48; G01N 33/50

(52) U.S. Cl. 435/6; 702/20

(57) ABSTRACT

Method for the identification of genes and genetic signals based on the structural properties of DNA double-helix comprising the following steps:

A—using the classical physical model of helix-coil transitions,

B—calculating stability curves (probabilities of opening of the DNA double-helix, along a given sequence) by algorithmic methods),

C—determining the disruption in the linear DNA for different temperatures,

D—analyzing the stability curves for the detection of genetic signals (disruption of the double-helix) or the identification of coding regions (simple genes or exons in split genes as regions of high thermal stability), and optionally,

E—based on the structural informations, performing classical sequence analysis (donor/acceptor sites, start and codon stops, in correspondence with the frontiers identified in the stability curves and open reading frames analyses) for completing the identification of genes.

### CROSS-REFERENCE TO RELATED APPLICATIONS

This application is based on and claims the benefit of U.S. Provisional Application Ser. No. 60/232,146, filed Sep. 13, 2000 (Attorney Docket No. 03495.6057). The entire disclosure of this Provisional application is relied upon and incorporated by reference herein.

### BACKGROUND OF THE INVENTION

The discovery that DNA is a double helix (Watson and Crick, 1953) represents the operative concept on which modern molecular biology and genetics were built. The differential, sequence-specific, propensity for the disruption of the DNA double-helix is described by the helix-coil transition model (see, for example: Poland and Scheraga, 1970; Cantor and Schimmel, 1980; Grosberg and Khokhlov, 1994). The separation of the two DNA strands is of critical importance in various processes such as transcription or replication, leading to the idea that genetic information (coding regions) and physical stability of the DNA helix may be intimately related. Analysis of this question had to await both the development of appropriate calculation methods and the availability of DNA sequences. The two conditions were met by the end of the seventies and the beginning of the eighties. The analysis of the sequences which were available at the time (essentially a few plasmids and phages, see, for example: Wells et al., 1980; Wada et al., 1980; Gotoh and Tagashira, 1981b; Gotoh, 1983; Suyama and Wada, 1983) provided no clearcut evidence for or against a relationship between the functional organization of the genetic message and the fundamental physical properties associated with the double-helical structure of DNA.

Now, in the genomics era, a very large number of long sequences (including complete genomes) is available. It is then mandatory to revisit the original question with this new information. It is precisely the object of the work here to present such a large-scale analysis of the genomic data (representing tens of millions base pairs as compared to the few tens of thousands base pairs involved in the previous analyses).

The implementation of structural models at the level of large genomic sequences is not a trivial task. The handling of structural models is hampered by a major methodological drawback concerning the long-range effects, which it must be taken into account in any realistic model. Long-range effects bring into ‘close contact’ elements which are distant on the primary sequences. In almost every case such effects lead to unaffordable calculation times, even for short sequences. Of course the situation becomes even more difficult when very long genomic sequences are to be treated. If we consider only nearest-neighbour interactions then the complete Coli sequence, with more than 4.6 millions base pairs, can be treated in seconds or minutes on a workstation. With the simplest possible long-range effect, such as the one involved in the helix-coil model for linear molecules, the calculation times are of the order of days or weeks for a phage 50,000 base pairs long (for a complete denaturation map). For still more complex models involving several mutually coupled long-range effects, such as in realistic models of helix-coil transitions in supercoiled molecules, the calculation times shift to hundreds or thousands years for a mere plasmid 5,000 base pairs long.

Usually, the only solution for overcoming the obstacle of long-range effects is to resort to more or less drastic approximations in the physics of the interactions (coarse-graining, meanfield representations, etc). In such a general methodological background, it should have been all the more remarkable that in one particular case an alternative solution was designed, tractable yet rigorous with respect to the physics. This solution (Poland-Fixman-Freim or ‘PFF’ algorithm (Fixman and Freire, 1977)) concerns precisely the helix-coil model in linear molecules. The long-range effect in this case is associated with the physical representation of the denaturation ‘bubbles’ (the basics of the model are recalled below). With the PFF algorithm it is possible to reduce the algorithmic complexity of the model, with the long-range effect, to complexities relevant to models of nearest-neighbour interactions. The only approximation in the formulation concerns the numerical representation of the long-range effect as a sum of exponential functions. The PFF (representing the culmination of more than 20 years of methodological developments) is expressed as a specialized algorithm specific to the model considered (recurrence relations for certain conditional probabilities). Probably for this reason there were no further generalisations to this approach. Also, various available programs implementing the PFF appear to be limited to sequences 1,000 base pairs long (‘POLAND’ program (Steger, 1994) or ‘MELT94’ program (http://web.mit.edu/osp/www/melt.html)), possibly to avoid overflow problems.

In such a context, the background of the invention concerns the problem of the ab initio identification of genes (notably in complex eukaryotic genomes), based on the implementation of the helix-coil structural model at the level of large genomic sequences. More precisely, the identification of genes relies most often on homology information. When such information is not available (or incomplete), the problem of ab initio gene identification can be very difficult, as illustrated notably by the recent debates concerning the potential number of genes in the human genome. With such a background, the specificity of the invention is to elaborate an ab initio gene identification method, which relies on the structural properties of the DNA (helix-coil transition model).

### SUMMARY OF THE INVENTION

Here the structural calculations (helix-coil model) on very long sequences (such as whole chromosomes) are performed using an algorithm called SIMEX. For the linear model, in terms of calculations, this SIMEX strictly reduces to the PFF (Yeramian et al., 1990). The SIMEX was nevertheless formulated on different conceptual grounds than the PFF, based on generic—explicit—evaluations of the partition functions. In this framework it appears that: (i) the representation of long-range effects (those which depend only on the number of elements in a given state, such as coiled or helical) as sums of exponentials is not a mere numerical trick proper to the helix-coil model but represents the only possible conceptual solution to accelerate drastically the calculations without oversimplifying the physics; (ii) the overflow problems are trivially solved, independent of sequence length; and (iii) the concepts can be generalized to higher-order models leading to calculations which can be accelerated up to millions folds (Yeramian, 1994). Ultimately, the overall effectiveness of such treatments implies that appropriate numerical representations must be obtained for the relevant long-range effects. The analysis of multiexponential functions, in general, is reputed to be a tough numerical problem. The Padé-Laplace formulation (Yeramian and Clayerie, 1987) provides an adequate solution to this problem. In what follows the helixcoil calculations are prefomed with the combined use of the SIMEX (for linear models) and Padé-Laplace methods.

Before performing the analyses at the genomic level it is necessary to illustrate the consequence of the inclusion of the long-range effect in the helix-coil model. This effect introduces a very sharp localization in the peaks in the stability maps, which plot the probability (along the sequence) for a base pair to be in the coiled state. Despite the fact that the helix-coil model is the most classical, archetypal, model in structural molecular biology such an illustration does not exist in the large literature devoted to the subject.

Stability maps are derived for various available genomes. It appears that for certain organisms the correlations between the genetic and physical stability maps is striking, almost perfect (even for very short genes such as those of tRNAs). It is then natural to suppose that there must be a functional meaning for such intimate correspondences between the two maps. As in other organisms such correlations do not exist at all, it is also necessary to suppose that the possible functional role is not of universal relevance. As such, the most plausible interpretation is that the correspondence between the two maps (when it is observed) reveals the relics of an archaic functional organization of the genetic information, possibly before the emergence of modem transcription machineries.

Based on the above conclusions and observations, it appears that the physical model of helix-coil transitions can be used for the practical purpose of gene identification, for the genomes in which a correlation is observed between coding regions and regions of relative high thermal stability. Such a situation is observed in a large series of eukaryotic genomes (Plasmodium falciparum, Drosphila melanogaster, Homo sapiens, Anopheles gambiae, etc.). In such cases, the high-stability regions appear to correspond to the coding regions (either simple genes or exons in split genes). As such, the physics-based gene identification (PBGI) method described here (based on the results of helix-coil transition curves), allows the ab initio identification of complex genes. This identification (concerning simple genes and exons in split genes) does not resort to any additional information concerning sequence homology, etc. The gene identification procedure elaborated on this basis is illustrated in detail in the case of Plasmodium falciparum, with notably the experimental confirmation for the predictions. The treatments are extended to the ab initio identification of genes in other eukaryotic genomes (Homo sapiens, Anopheles gambiae, etc.). The PBGI scheme also allows to detect genes, which are transcribed but not translated.

### BRIEF DESCRIPTION OF THE DRAWINGS

This invention will be described in detail with reference to the following drawings:

FIG. 1. Classical model of helix-coil transitions in linear DNA.

The physics of the transitions is represented schematically, with increasing temperatures. Disruptions in the double-helical structure occur at the extremities of the linear molecule (denaturation from the extremities) as well as within the molecule. Single-stranded loops appear at specific sites, depending on the precise sequence. Because of the cooperativity of the transition, when two loops are close enough they tend to merge together.

FIG. 2. Helix-coil calculations for the whole yeast chromosome VIII.

All treatments are for the complete sequence of yeast chromosome VIII, as available in the MIPS databank (http://www.mips.biochem.mpg.de/proj/yeast/).

(A) GC % plot. The calculation is done with a 500 base pairs sliding window.

(B) Stability map with the nearest-neighbour model. The probability for a base pair to be in the coiled state is plotted along the sequence for the temperature T=63° C.

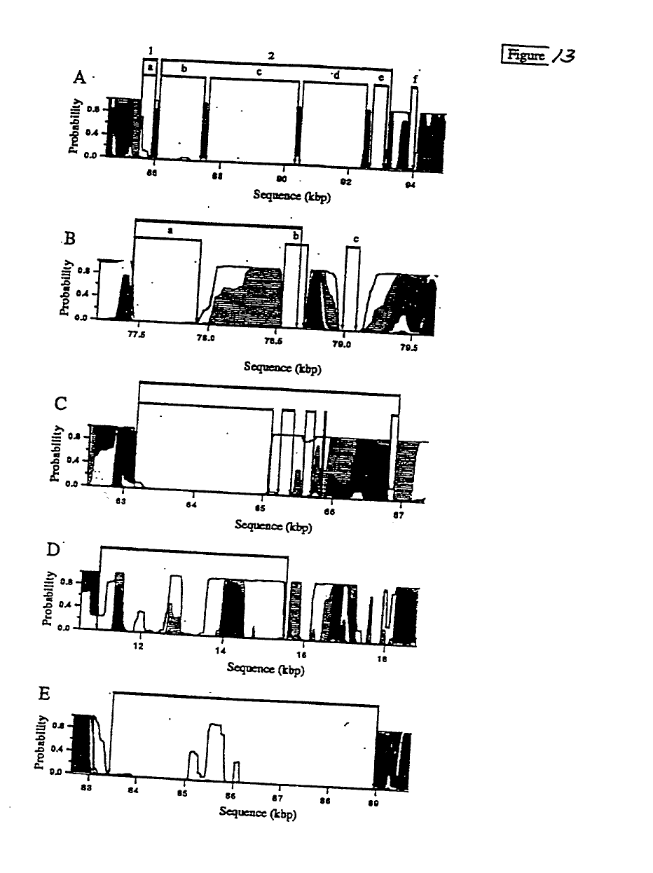

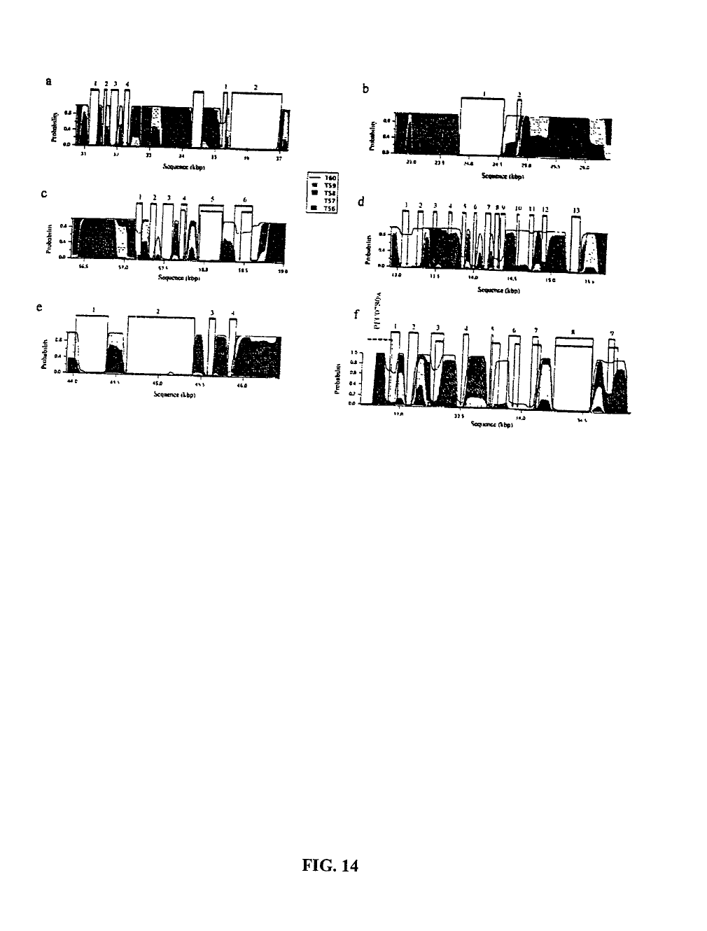

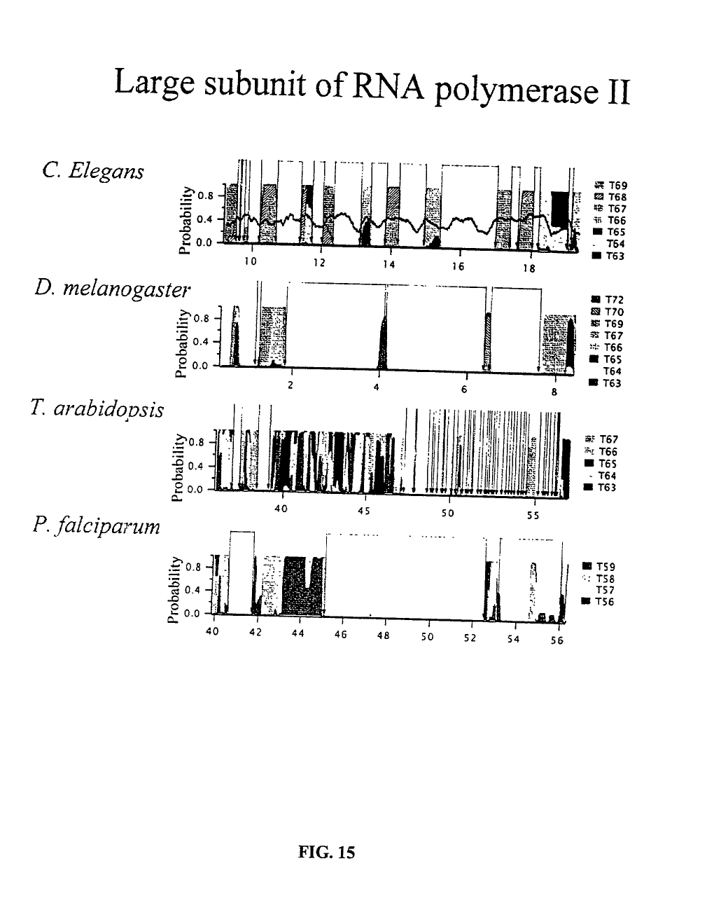

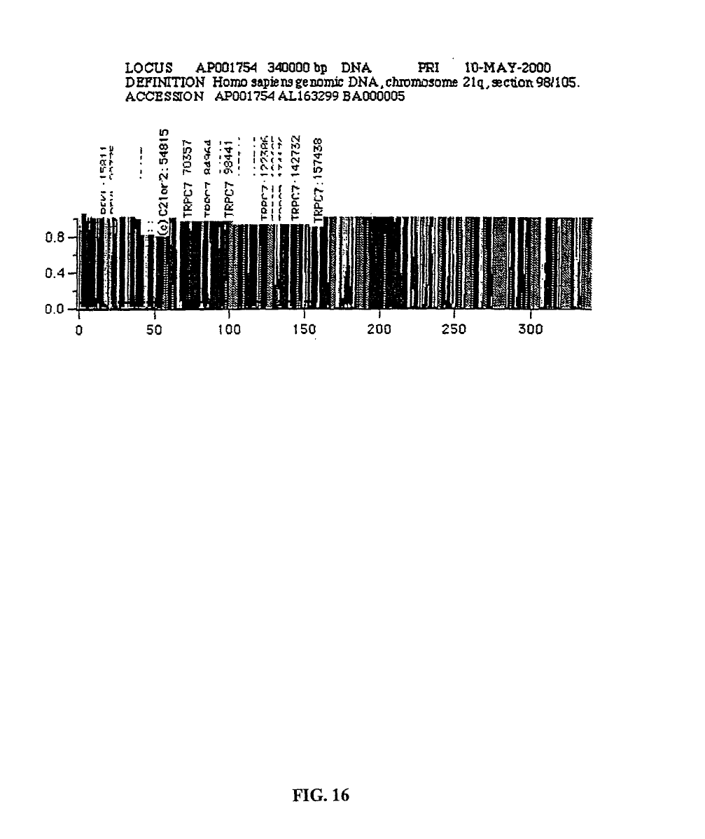

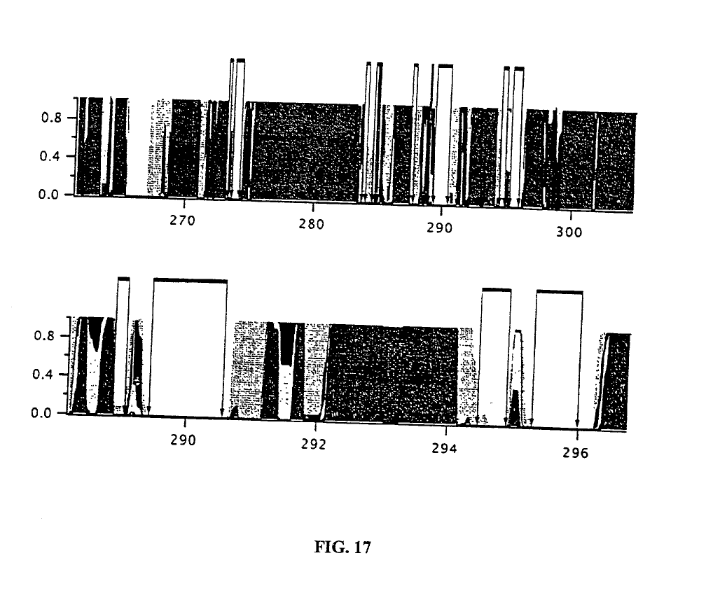

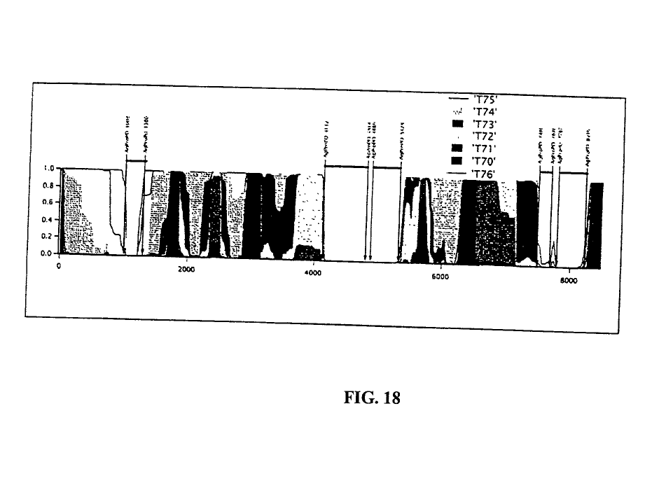

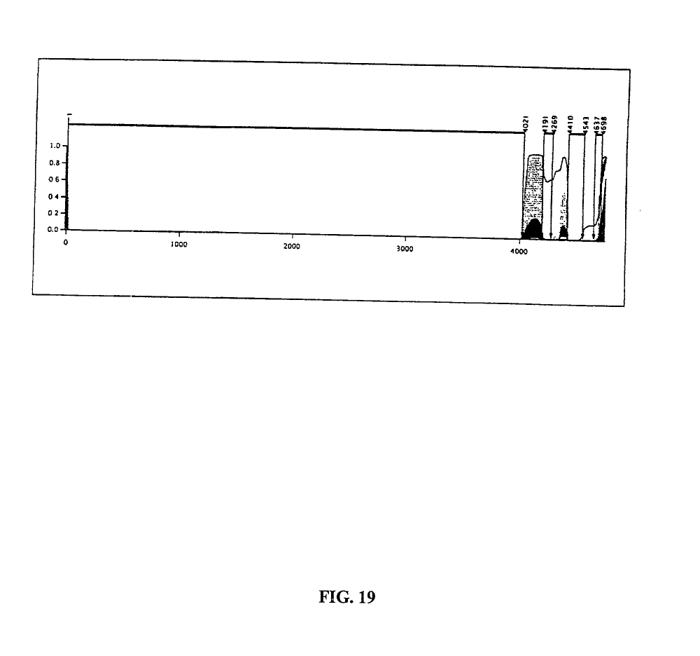

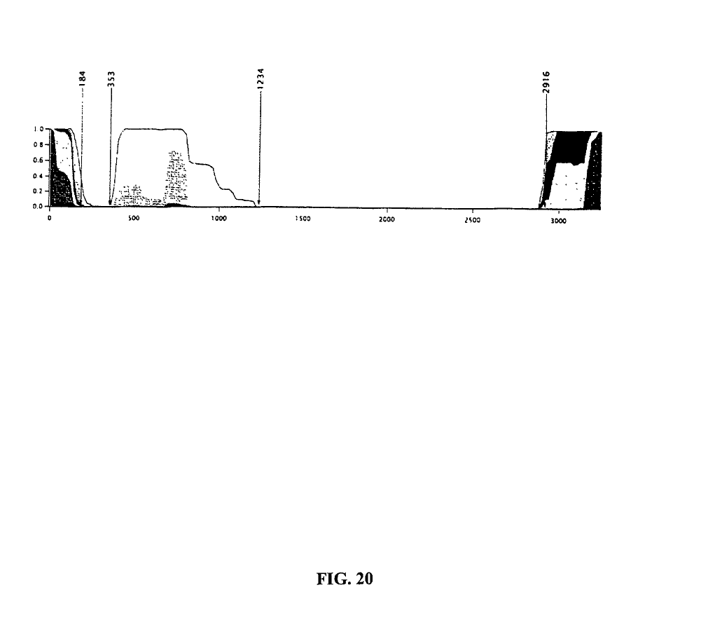

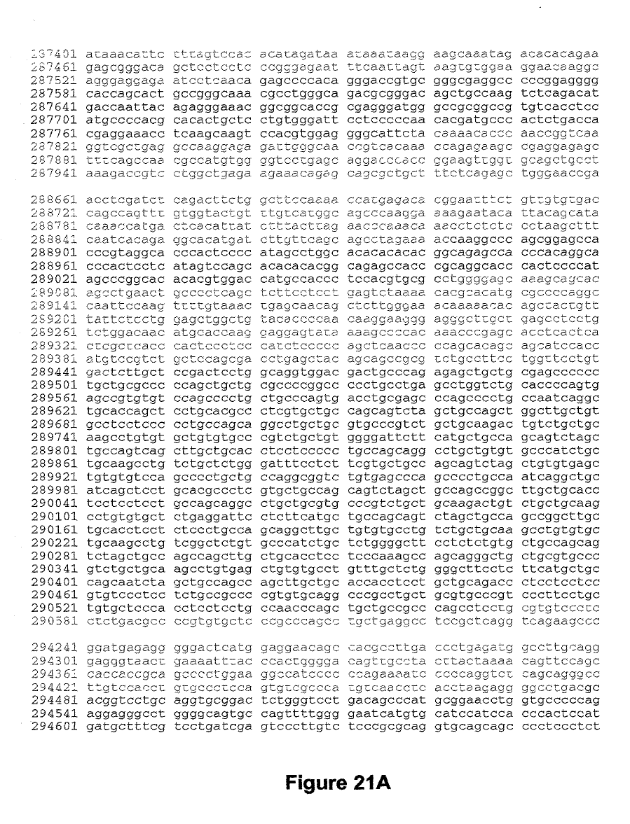

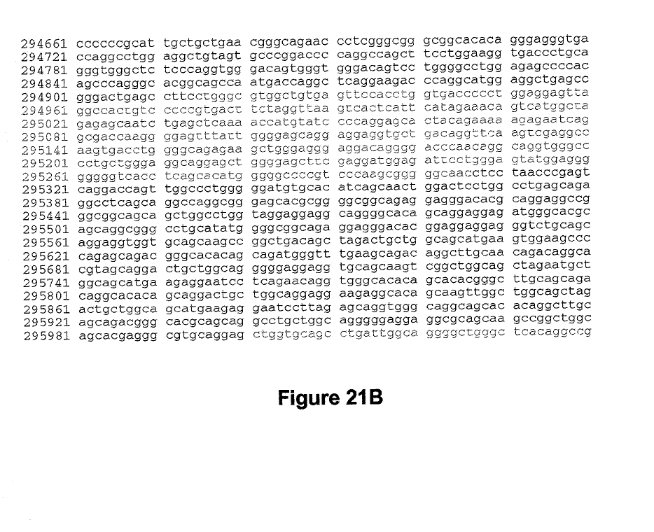

(C) Stability map with the long-range effect. The helix-coil calculations are performed with exactly the same parameters, and the same temperature, as in FIG. 2B with the only difference being that it is taken into account of the length-dependent part of the loop-entropy weight.

(D) Close-up for the detailed stability map with 5 different temperatures. With the same conditions as in FIG. 2C, the stability maps for 5 different temperatures are superposed (63° C.-67° C.) and a close-up of the corresponding composite map is shown for the chromosonic region extending from 100,000 to 140,000 base pairs. The color coding for the temperatures is displayed with T63, for example, standing for T=63° C. (this convention is adopted throughout the figures).

(E) Superposition of stability and genetic maps. A chromosonic region of 20,000 base pairs long is shown (from 100,000 to 120,000 base pairs), with the positions and orientations of the 8 genes in the region indicated by dark blue arrows. The two superposed stability maps displayed were calculated as in FIGS. 2B and 2C, for the temperature T=68° C., without (cyan blue) and with (magenta) the loop-entropy long-range effect.

FIG. 3. Stability maps for Prototheca wickerhamii and Mycobacterium tuberculosis sequences.

(A) Stability map for the Prototheca wickerhamii complete mitochondrial genome. For this genome (Accession number: U02970) the stability map, with 4 different temperatures, is displayed from position 10,000 to 20,000 base pairs. The genes as documented in the annotation are reported, superposed on the stability map, as blue arrows, with the names of the genes. A series of 14 tRNA genes are also reported, coded with the purple color.

(B) Stability map for the Mycobacterium tuberculosis complete genome. For this genome (http://www.sanger.ac.uk/Projects/M_tuberculosis/ or http://bioweb.pasteur.fr/GenoList/TubercuList/) the stability map, with 6 different temperatures, is displayed for a 30,000 base pairs long region. For this randomly chosen region, the origin was set arbitrarily (the beginning of the first gene displayed—Rv1331—corresponds to the position 1,500,659 base pairs in the annotation of the genome).

FIG. 4. Stability properties of a duplicated region in the yeast genome.

Sequences and annotations are as in the MIPS database.

(A) Stability map, for a duplicated region of yeast (block 9 in chromosome III). The stability map, with 5 different temperatures, is displayed up to the gene YCL035c of this block. (B) Stability map, with ‘high’ temperatures, for the sequence shown in FIG. 4A. The stability map is displayed with 9 different temperatures (4 temperatures in addition to those shown in FIG. 4A). (C) Stability map for a portion of the duplicated block 9 in chromosome IV. The stability map is displayed for the same temperatures as in FIG. 4A. Gene YDR516c (green arrow) is homologous to gene YCL040w and YDR518w (red arrow) is homologous to YCL043c. (D) Stability map, with ‘high’ temperatures, for the sequence in FIG. 4C. The 9 temperatures are the same as in FIG. 4B. (E) Stability map for the gene YGL235w in chromosome VII. This gene is homologous to YCL040w, and YDR516c.

FIG. 5. Stability properties for tandemly duplicated genes ir the yeast genome.

Sequences and annotations are as in the MIPS database.

(A) Stability map for the tandemly duplicated genes YDR038c, YDR039c and YDR040c, in chromosome IV. (B) Stability map for the tandemly duplicated genes YAR027w to YAR033w in chromosome I. The tandemly duplicated genes are represented with pale orange arrows. (C) Stability map, with ‘high’ temperatures, for the sequence in FIG. 5B.

FIG. 6. Snapshots for stability curves from the chromosome 2.

For various conditions used for the calculations, and conventions, see the text. The stability curves are plotted for 5 temperatures (56° C. to 60° C.). The colour coding for the temperatures is displayed with T56, for example, standing for T=56° C. The plots in black, or red (panel D), correspond to the GC % curves (the y-axis is scaled such as the range [0-1] for the probabilities corresponds to the range [0%-50%] for the GC composition). The GC % curves are calculated with a sliding window of 100 base pairs long (plots in black) or 200 base pairs long (plots in red). Coding regions (as predicted in the database annotation) are represented by red and blue horizontal bars (the alternance of the two colours; is used to clearly distinguish a gene from the previous and next ones). The names of the genes (as in the database annotation of the complete chromosome, Gardner, 1998) are reported above the horizontal bars indicating the putative coding regions.

(A) Stability maps for a sequence from chr2(0_{—}100). (B) Stability maps for a sequence from chr2(200-300). (C) Stability maps for a sequence from chr2(300_{—}400). (D) Close-up views for two regions from the stability maps in (C).

FIG. 7. Analysis of cloned genes.

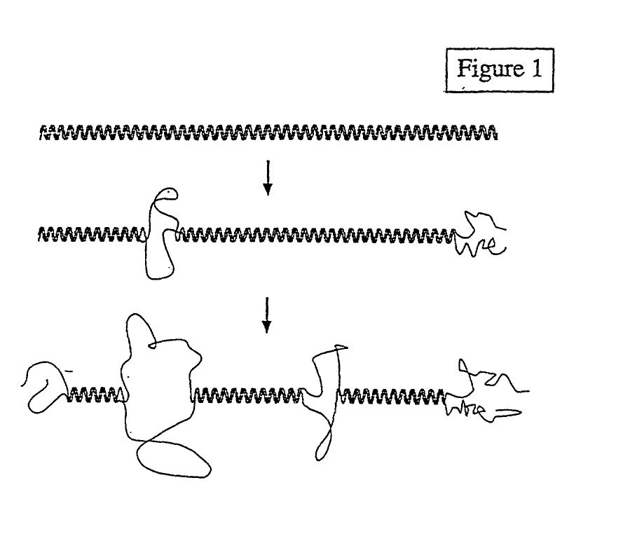

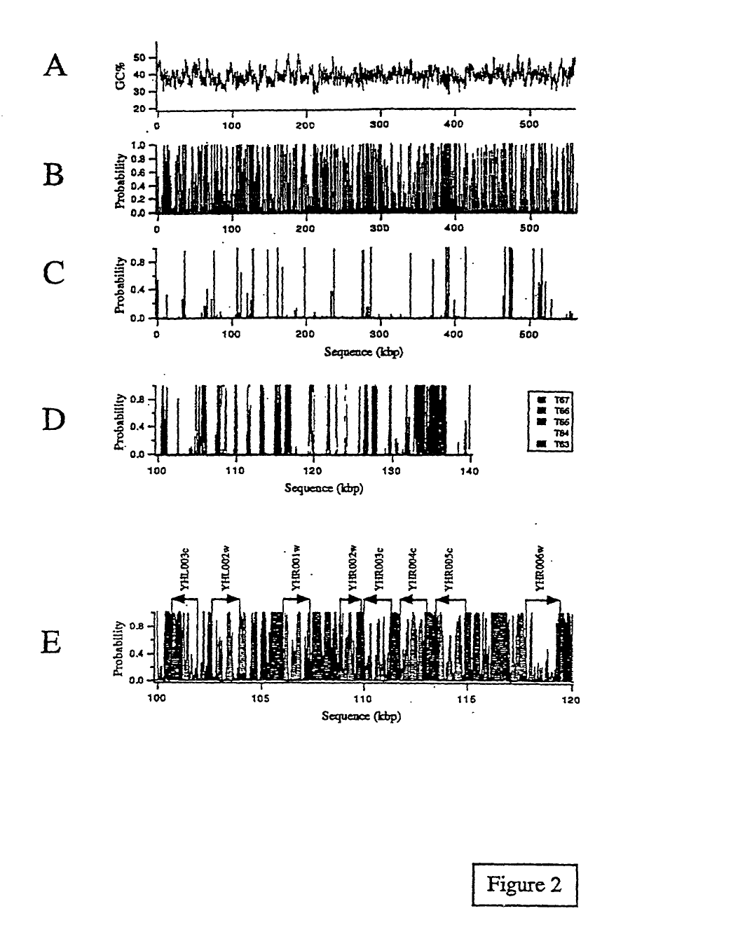

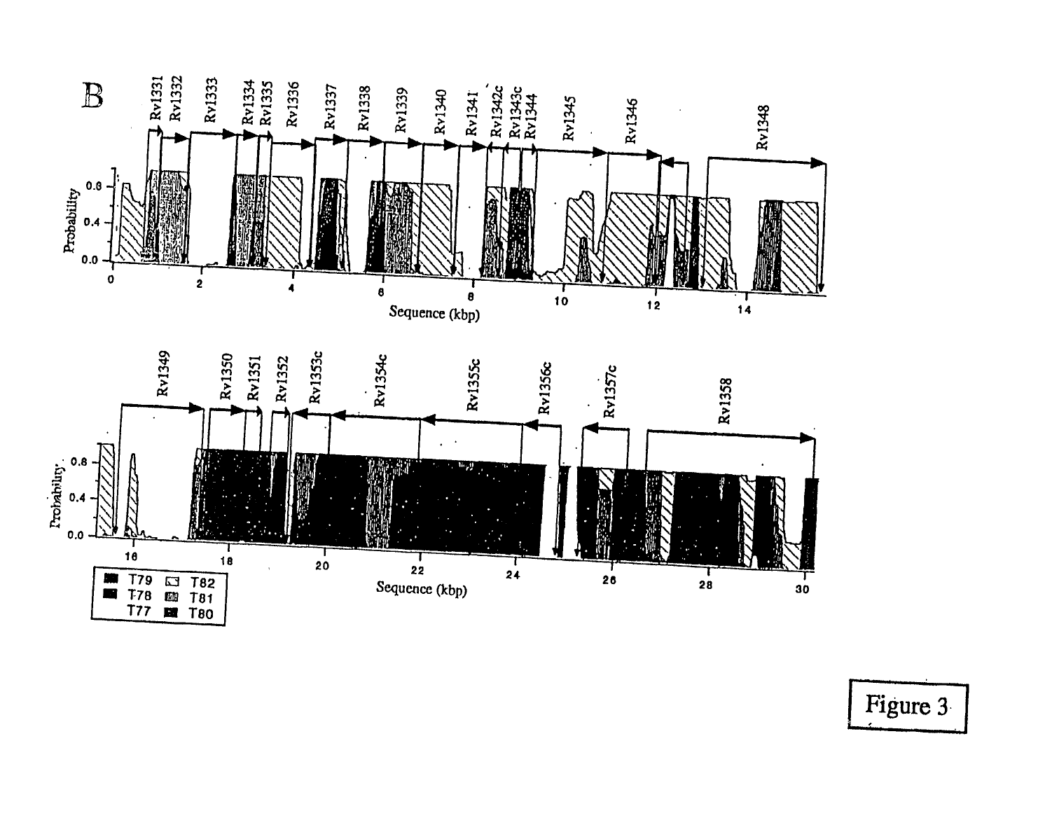

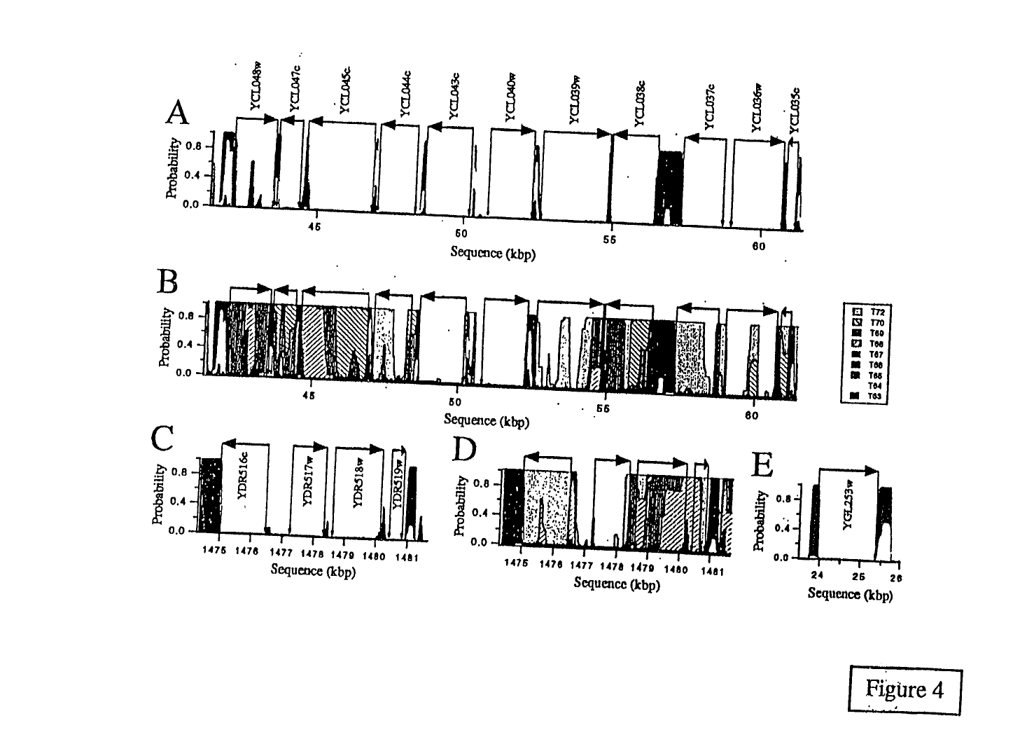

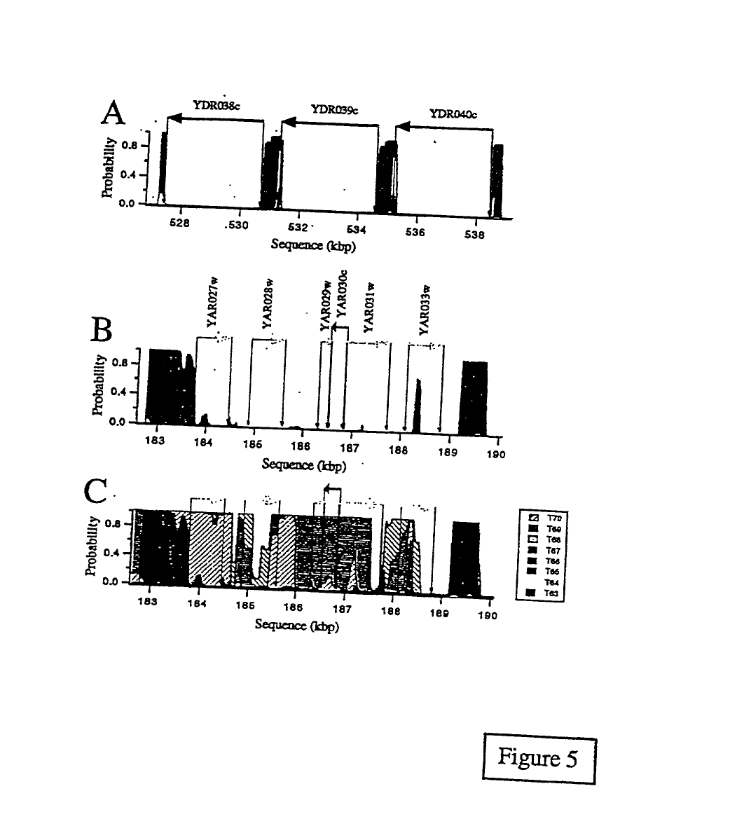

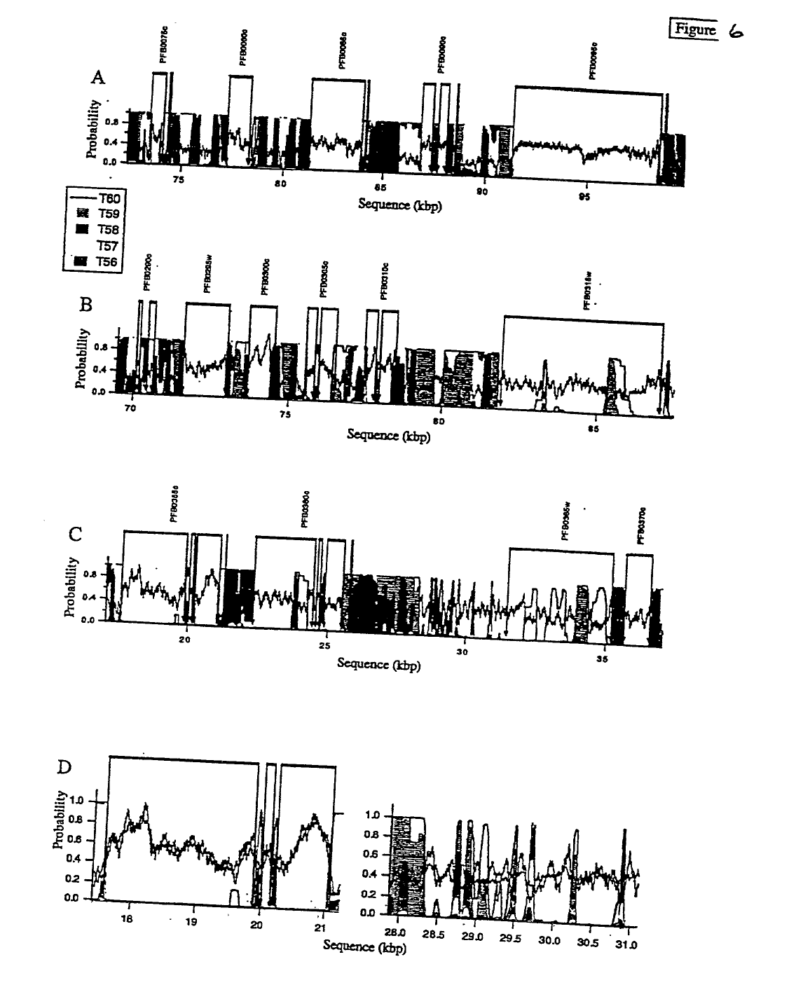

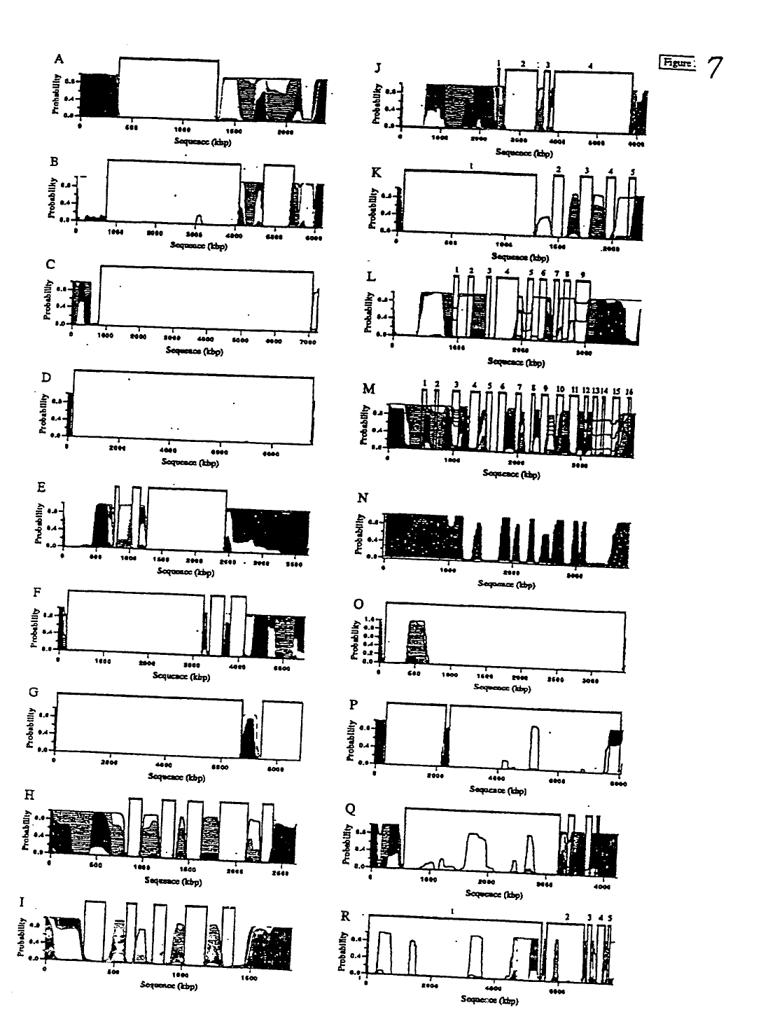

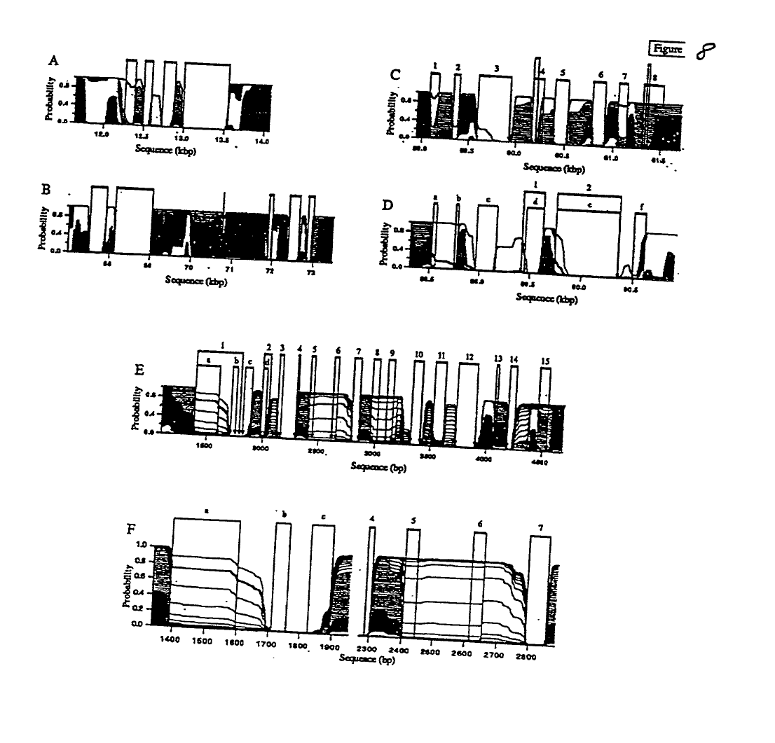

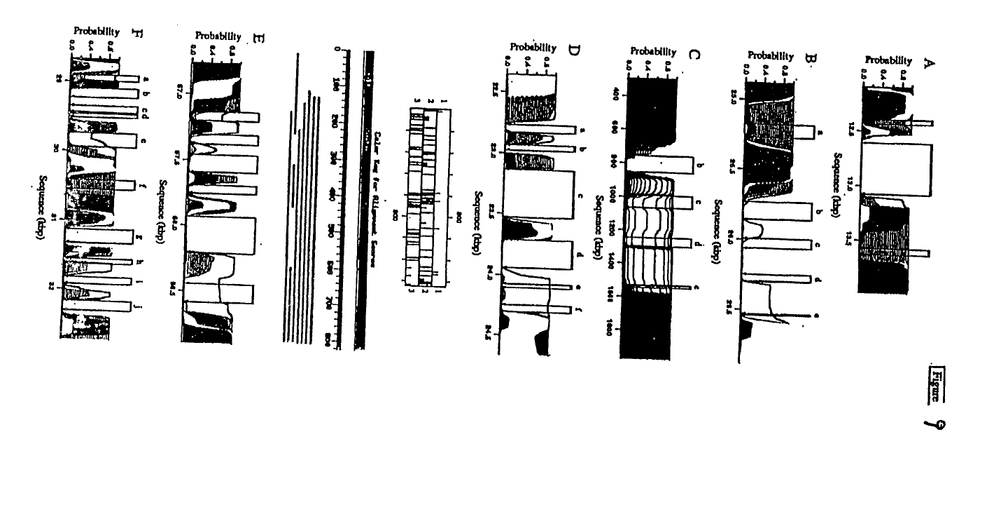

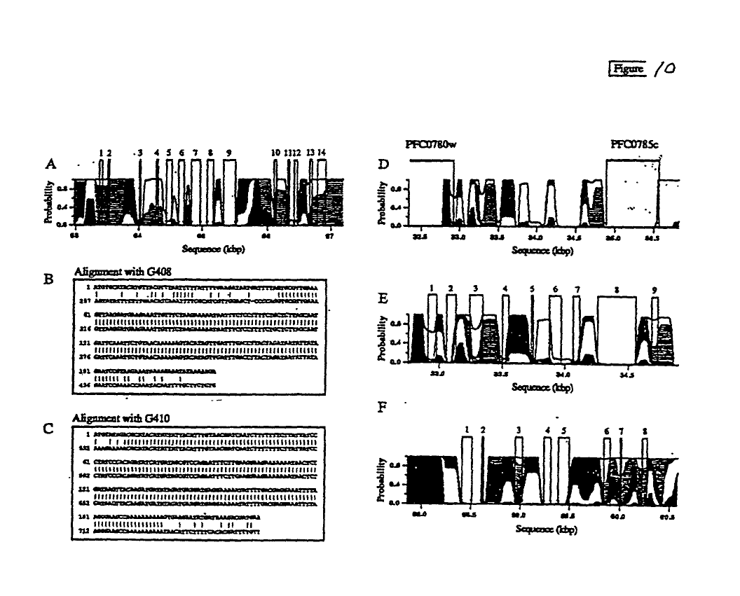

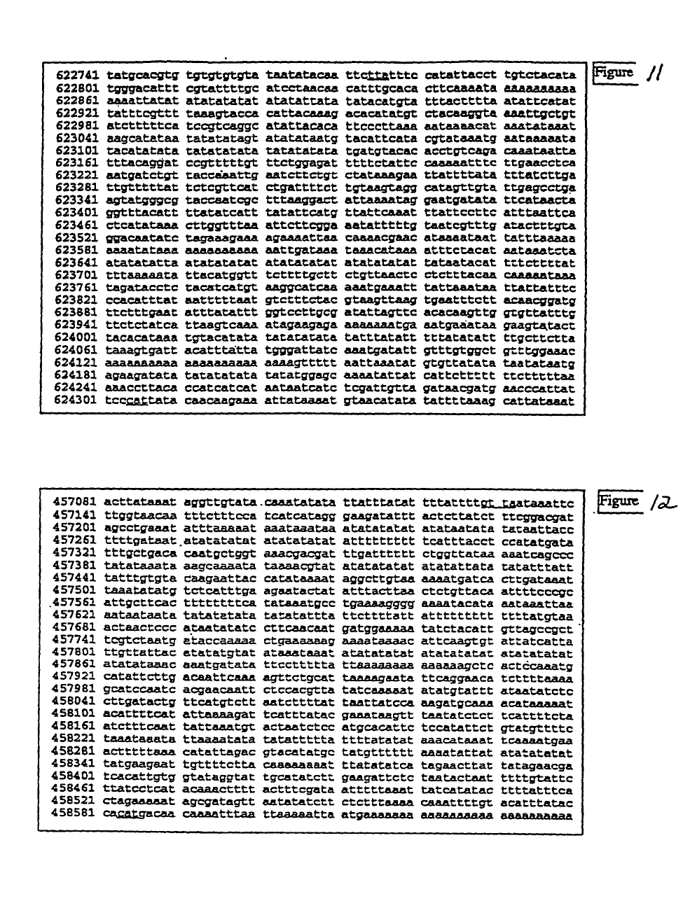

Stability maps are represented with the conventions of FIG. 1 (unless otherwise specified).

(A) A gene encoding the mitochondrial phosphate carrier (Bhaduri-McIntosh and Vaidya, 1996; accession: U49381). (B) Gamma-glutamylcysteine synthetase gene (‘GCS’ gene, in blue; Luersen et al., 1999; accession: AJ006966), with nearby another simple gene in the same sequence (in red). (C) CTRP gene (Trottein et al., 1995; accession: U34363). (D) Pfs230 gene associated with transmission-blocking target antigen (Williamson et al., 1993; accession: L08135). (E) Alpha-tubulin II gene (Holloway et al., 1990; accession: M34390). (F) Ca(2+)-ATPase gene (Kimura et al., 1993; accession: X71765). (G) A var gene (Reeder et al.; accession: AF 134154). (H) Pfc2 gene associated with a protein kinase (cdc2-like protein kinase; Ross-Macdonald et al., 1994; accession: X61921). (I) Arf gene for ADP-ribosylation factor (Stafford et al., 1996; accession: Z80359). (j) A ‘SERA’ gene (Fox and Bzik, 1994; accession: U08113). (K) cpk (kinase) gene (Zhao et al., 1993; accession: X67288). (L) Blood stage antigen (41-3) gene (Knapp et al., 1991; accession: M59961). In addition to the standard conditions the stability maps associated with T61 and T62 are drawn as red lines. Alternative splicing has been demonstrated for this gene, yielding three different mRNAs (in addition to the one corresponding to the 9 exons, the following two combinations: 1+2+3+4+8+9 and 1+2+3+4+6+8+9). (M) Primase small subunit gene (Prasartkaew et al., 1996; accession: X99254). The cyan lines correspond to the stability maps associated with the temperatures T59.5 to T59.9, by steps of 0.1° C. (N) The sequence is the same as in (M), and the two stability maps are associated with the temperature T59.7. The dark purple line corresponds to calculations with interpolations (probabilities evaluated every 20 base pairs) whereas the filled plot in light purple corresponds to calculations without interpolations. (0) PfPK4 gene (eIF-2alpha kinase-related enzyme, Mohrle et al., 1997; accession: X94118). A coherent ORF-analysis can be performed with the low-stability region assimilated to an intron (with the original annotation in X94118 replaced by: join(69 . . . 388, 698 . . . 3440)). (P) A gene whose product is thought to be associated to an exported serine/threonine protein kinase (Kun et al. 1997; accession: U40232). (Q) Para-aminobenzoic acid synthetase gene (Triglia and Cowman, 1999; accession: AF 119554). (R) RNA polymerase III largest subunit gene (Li et al., 1991; accession: M73770).

FIG. 8 Genes from chromosomes 2 and 3 with known similarities.

Conventions as in FIG. 1 (unless otherwise specified). All exons in green correspond to rectifications or new predictions, suggested by the physics. For rectifications, or new predictions, the coordinates are provided with the same conventions as in the database annotations. For example ‘join(n1 . . . n2, n3 . . . n4)’ corresponds to a gene with two exons (n1 to n2, and n3 to n4, respectively), whereas for a gene in the reverse direction the notation ‘complement(join(n1 . . . n2, n3 . . . n4))’ is used.

(A) PFC0865w gene (MAL3P7.2, similar to C. elegans RNA-binding protein) in chr3(800_{—}900). (B) PFC0915w (MAL3P7.12, similar to ATP-dependent RNA helicase) and PFC0920w (MAL3P7.13, similar to C. elegans histone H2A variant) genes in chr3(800_{—}900). The very small exon in green (870762 . . . 870768) is appended to the second gene (PFC0920w). (C) PFB0505c gene (similar to 3-ketoacyl carrier protein synthase III) in chr2(400_{—}500). The rectifications in green replace the exon (460162 . . . 460275) by (460162 . . . 460206) and the exon (461335 . . . 461518) by (461343 . . . 461382). (D) PFB0425c gene (similar to yeast YMR7 gene) in chr2(300_{—}400). The plot in red corresponds to T61. The annotation in green corresponds to a gene with 6 exons (a to f), with coordinates: join(complement(388524 . . . 388560, 388755 . . . 388784, 388964 . . . 389162, 389448 . . . 389618, 389742 . . . 390349, 390500 . . . 39061 1)). (E) PFC0495w gene (similar to E. tenella aspartyl protease) in chr3(400_{—}600) (the gene overlaps the 400_{—}500 and 500_{—}600 stretches, following the conventions here). Calculations are performed (without interpolations) on a sequence extending from positions 498001 bp (taken as the origin for the stability curves in the figure) to 503580 bp, of the chromosome sequence. The magenta lines correspond to 9 temperatures (59.1° C. to 60.1° C., by steps of 0.1° C.), in addition to the 5 routine temperatures. The rectifications in green concern the exon 1, replaced by exons a, b and c (coordinates (499392 . . . 499598, 499711 . . . 499755, 499824 . . . 499893)), and the exon 2, replaced by exon d (coordinates (499985 . . . 500023)). (F) Two close-up views of the stability maps in (E).

FIG. 9. Discovery and annotation of new putative genes in the chromosome 2.

Conventions as in FIG. 1 (unless otherwise specified). Annotations as in FIG. 3.

(A) New putative gene in chr2(100_{—}200), designated as PFB0107c. complement(join(112383 . . . 112432, 112612 . . . 113075, 113576 . . . 113633)) (B) New putative gene in chr2(400_{—}500), designated as PFB0467w. The annotation for this gene is: join(425181 . . . 425272, 425733 . . . 425855, 425995 . . . 426065, 426248 . . . 426299, 426531 . . . 426543). (C) Stability maps for the same sequence as in (B). The probabilities are evaluated every base pair and the origin is set at the first base pair of a 2.7 kb sequence which spans the predicted gene. The plain curve in blue corresponds to the condition T59.2, and all the red lines correspond to the conditions T59.3 to T60, by steps of 0.1° C. (D) New putative gene in chr2(600-700), designated as PFB0687c. The annotation for this gene is: complement(join(622777 . . . 622840, 622939 . . . 622982, 623139 . . . 623529, 623715 . . . 623944, 624073 . . . 624108, 624250 . . . 624306). The detailed exon-assembly at the sequence level is displayed in FIG. 6. The outputs for the ORF-analysis and Blast searches (Blastx, with the color keys for the alignment scores corresponding to the NCBI inventions) are displayed below the stability plots. (E) New putative gene in chr2(400_{—}500), designated as PFB0503c. The annotation for this gene is: complement(join(457133 . . . 457203, 457309 . . . 457379, 457461 . . . 457589, 457687 . . . 457744, 457933 . . . 458208, 458447 . . . 458585)). The detailed exon-assembly at the sequence level is displayed in FIG. 7. (F) New putative genes in chr2(700_{—}800), designated as PFB0827c (exons a to j). The annotation for this gene is: complement(join(728915 . . . 728995, 729110 . . . 729239, 729359 . . . 729448, 729473 . . . 729525, 729744 . . . 729941, 730413 . . . 730544, 731135 . . . 731331, 731548 . . . 731623, 731818 . . . 731921, 732171 . . . 732312)).

FIG. 10. Discovery of new putative genes in the chromosome 3.

Conventions as in FIG. 1.

(A) New gene in chr3(500_{—}600), designated as PFC0585w. The annotation for this gene is: join(563377 . . . 563425, 563508 . . . 563537, 564010 . . . 564034, 564276 . . . 564305, 564434 . . . 564520, 564632 . . . 564714, 564830 . . . 564966, 565077 . . . 565174, 565321 . . . 565511, 566109 . . . 566142, 566316 . . . 566337, 566420 . . . 566467, 566654 . . . 566694, 566786 . . . 566921). (B) Alignment between the coding sequences of the PFC0585w gene (lower sequence) and the G408 gene (upper sequence), see text. (C) Alignment between the coding sequences of the PFC0585w gene (lower sequence) and the G410 gene (upper sequence), see text. (D) Genomic region in chr3(700_{—}800), between the genes PFC0780w (MAL3P6.15, in red, with the gene further extending on the left-side of the figure) and the gene PFC0785c (MAL3P6.16, in blue). (E) Annotation of the exons associated with the stability plots in (D). The 9 exons are appended to PFC0780w, whose new annotation becomes: join(724949 . . . 732808, 732909 . . . 732980, 733057 . . . 733133, 733240 . . . 733345, 733503 . . . 733549, 733733.333747, 733875 . . . 733968, 734067.734116, 734257 . . . 734556, 734685 . . . 734737). (F) New gene in chr3(700 800), designated as PFC0813c, between the genes PFC0810c (MAL3P6.21) and PFC0815c (MAL3P6.22). The annotation for this gene is: complement(join(758414 . . . 759512, 758615 . . . 758635, 758952 . . . 759027, 759242 . . . 759311, 759390 . . . 759500, 759840 . . . 759907, 760005 . . . 760017, 760215 . . . 760274, 760475 . . . 760525)).

FIG. 11. Detailed exon-assembly for the gene PFB0687c.

Sequence-anlysis for the exon-intron structure of the PFB0687c gene, as represented. graphically in FIG. 4D. Exon sequences are represented in blue and intron sequences in green (the rest of the genomic sequence, nor relevant for the analyzed gene, is in black). Start and stop codons (underlined) as well as splice signals are represented in magenta.

FIG. 12. Detailed exon-assembly for the gene PFB0503c.

Sequence-analysis for the exon-intron structure of the PFB0503c gene, as represented graphically in FIG. 4E. Conventions as in FIG. 6.

FIG. 13. Low-stability regions within large open reading frames in genes from chromosomes 2 and 3.

Conventions as in FIG. 1. Annotations as in FIG. 3.

(A) Stability maps associated with the gene PFC0485w. The two exons corresponding to the database annotation are in blue. A new annotation (6 exons, a to f, in green) is also represented. This annotation takes into account of an additional exon at the right-end of the sequence and assimilates the three sharp low-stability regions (within the second exon in blue) to introns. The corresponding new annotation is: join(485498 . . . 485941, 486080 . . . 487423, 487592 . . . 490361, 490491 . . . 492454, 492671 . . . 493123, 493849 . . . 494042). (B) Stability maps associated with the gene PFB0530c. The simple gene of the database annotation is represented in blue. A possible rectification for this annotation is represented with three exons in green. The new annotation is: complement(join(477435 . . . 477913, 478538 . . . 478704, 478991 . . . 479079)). (C) Stability maps associated with the gene PFB510w. The simple gene as corresponding to the database annotation is in blue. A possible alternative annotation is also represented, with exons in green. A complete gene-assembly as based on this solution is not performed. (D) Stability maps associated with the gene PFC0415c. (E) Stability maps associated with the gene PFB0540w.

FIG. 14. Experimental confirmation for the physics-based gene predictions (Plasmodium falciparum)

The probability of helix opening is calculated along the genomic sequences (chromosome 2 in 14a to 14e, chromosome 3 in 14f for various temperatures (T56 for example standing for the temperature 56° C, the temperatures are relative to standard energetic and thermodynamic parameters for the DNA double-helix (Yerarmian, E. Gene 255,139-150 (2000); Yeramian, E. Gene 255, 151-168 (2000)). The calculations are performed for stretches of 100 kbp (in 14a the origin is set at 600 kbp, in 14b at 800 kbp, in 14c at 400 kpb, in 14d at 600 kbp, in 14e at 500 kbp, and in 14f at 700 kbp). The stable regions are those which remain in the helical state (probability zero to be in the coiled state). The frontiers of the coding regions are shown by vertical arrows. The corresponding uninterrupted genes, or exons, are represented by horizontal bars (in different colors). Detailed annotations for the cloned genes are provided as supplementary information. (A) Genes PFB0827c (blue: a PBGI prediction confirmed by sequencing), PFB0830w (red: database annotation) and PFB0833c (green: database annotation for the long exon, the small exon corresponds to a putative missed exon). (B) Gene PFB0927c (blue: a PBGI prediction confirmed by sequencing), (C) Gene PFB0503c (a PBGI prediction confirmed by sequencing). This prediction is reported as FIG. 4E in Yeramian, E., Gene, 255, 151-168 (2000). When differences are observed between the experimental results (exons in blue) and the predictions (exons 4, 5 and 6), the predicted exons are drawn in green. The same conventions are adopted in FIG. 14f. (D) Gene PFB0683w (blue: a PBGl prediction confirmed by sequencing for the 5 first exons). (E) Gene PFB0612c (red: original database annotation, blue: exons predicted by PBGI and confirmed experimentally). (F) Gene PFB0780w, with the original annotation corresponding to a simple gene 7973 base pairs long, extending at the left-side of the graph (indicated as a dashed line in red). The 9 exons predicted by PBGI (FIG. 7E in Yeramian, E., Gene, 255, 151-168 (2000)) were confirmed by sequencing (exons in blue, also represented in green for the predictions, whenever differences are observed between predictions and experience).

FIG. 15. Physics-based analysis of the large subunit of RNA polymerase II.

FIG. 16. Analysis of a genomic sequence from H. sapiens (Accession No.: AP001754).

FIG. 17. Close-up view of FIG. 16.

FIG. 18. Gene identification for the gene AgProPO of Anopheles gambiae (Accession No.: AF031626).

FIG. 19. Physics-based gene analysis of a non-translated gene of Plasmodium falciparum.

FIG. 20. Physics-based analysis of the G6PD gene in Plasmodium falciparum (Accession No.: X74988).

### DETAILED DESCRIPTION OF THE INVENTION

2.1. Physical Model

The classical model of helix-coil transitions in linear DNA is represented schematically in FIG. 1. With increasing temperatures, the denaturation of DNA is a cooperative sequencedependent phenomenon leading to the progressive appearance of single-stranded regions. The physical bases of this model have been established and validated by experimental and theoretical studies (there is an extremely large literature devoted to this subject, see for example: Poland and Scheraga, 1970; Cantor and Schimmel, 1980; Grosberg and Khokhlov 1994; Wada et al., 1980; Gotoh, 1983; Lyubchenko et al., 1976; Lyubchenko et al., 1978; Wartell and Benight, 1985). The appropriate framework for the theoretical handling of the model is statistical mechanics (Poland and Scheraga, 1970; Cantor and Schimmel, 1980; Grosberg and Khokhlov 1994), leading to the evaluation of the partition function. Various quantities of interest associated with the model are readily evaluated from this function. Simplified representations are adopted in which there are two accessible states for a given base pair (either helical or coiled). Thus the model is relevant to the classical one-dimensional Ising model in physics, with nevertheless two notable differences: sequence-specificity and a long-range effect that concerns the properties of the internal loops (such as those shown in FIG. 1) as an important component in the cooperativity in the model. The sequence-dependence results from the crucial difference in physical stability of AT and GC pairs: the two bases in a pair are held together by two, or three hydrogen bonds, respectively. The physical representation of the loops is provided by polymer physics. For an internal loop with j base pairs in the coiled state, a weight is attributed (‘loop-entropy factor’) that relates to the ring-closure probability for a singlestranded coil of the same total length. This factor is written as σω(j), with σ a constant parameter for given conditions (‘helix nucleation’) and the length-dependent (long-range) part ω(j) written as a power-law ((2j+2)^{-α}, with 2j+2 single-stranded ‘elementary links’ for j open base pairs) as first derived in the Jacobson-Stockmayer theory (Jacobson and Stockmayer, 1950; Bloomfield et al., 1974). In a nearest-neighbour treatment the length-dependent part is completely neglected and the loop-entropy factor reduces to the constant helix nucleation term. Such a simplification is not realistic as it supposes that the ring-closure probability for a single-stranded DNA coil does not depend on the size of the coil. As a matter of fact, the long-range contribution in the loop-entropy factor plays an important role in the ability of the helix-coil theory to correctly account for the experimentally observed cooperativity in the transitions (qualitatively and quantitatively). More precisely, this cooperativity concerns the tendency of two separate (relatively small) loops to merge together, when they are close enough, to form one single larger loop. This tendency results from the energetic description of the second configuration (one large loop) being favoured (in a vast majority of cases) over the first one (two smaller loops), when the long-range contribution is taken into account (for a detailed quantitative illustration of this feature see the examples on page 1208 of Cantor and Schimmel, 1980).

2.2. Algorithms

If ω(j) is neglected, then the algorithmic treatment reduces to a nearest-neighbor model and the time needed for the evaluation of the partition function grows only proportionally with the length (n) of the sequence (time complexity in O(n)). In this case, in order to ensure the loop-closures, at any given step (i) of the processive calculation along the sequence, we only need to keep track of two configurational classes: those which end at position i−1 with an element in the helical state and those which end with an element in the coiled state. On the other hand, the time complexity of the complete model that takes into account of the length-dependent part is in O(n²). This time-complexity results from the fact that in this case we need to distinguish, at step i, i−2 configurational classes (based on the lengths of the coiled regions, ranging between 1 and i−2) for which the loop-closures must be ensured separately. Accordingly, the number of ring-closures (at each step of the processive treatment) becomes increasingly large with the length of the sequence.

In what follows, we plot along a given sequence, and for a given temperature, the probability that a base pair at a given position is in the coiled state. We shall call such a plot a stability map. For a sequence of length n, the calculation of a full stability map involves the evaluation of n partition functions. Accordingly, the overall time complexity for such an operation is O(n²) for the nearest-neighbour model and O(n³) for the complete model, including the long-range effect. Calculations in O(n³) are not manageable for large sequences. With the representation of the long-range effect as a multiexponential function, the SIMEX method allows the complexity for the complete model of helix-coil transitions to be reduced from O(n³) to O(n²) (with some constant prefactor depending on the number of exponentials in the multiexponential representation). The basic idea is to perform the various loop-closures in a single step, by merging the i−2 classes above into a single configurational class handled as in the nearest-neighbour model. In such a merging the attribution of weights to loops of different lengths is performed—processively—through local contributions added at each step, and required to add-up and give back the appropriate long-range global contributions. Such a condition can be fulfilled only by the exponential function, through its fundamental property (exp(i)exp(j)=exp(i+j)). For a general ω(j) function it is necessary to resort a to an accurate representation of the function with N exponential terms (with the property preserved for each one of them). In such a scheme, the number of operations involved at each step i will be constant (depending only on N), instead of increasing monotoneously with i (hence the reduction one order of magnitude in the complexity of the calculations).

The SIMEX is based on the explicit evaluation of the partition function, and, accordingly, a probability (such as the probability for a base pair to be in the coiled state) is expressed as the ratio of two partition functions. It is then straightforward to avoid any underflow (or overflow) problem through a mere normalisation between the numerator and the denominator (Yeramian et al., 1990). In the implementation the normalisation was performed every 50 base pairs.

2.3. Long-Range Effect and its Multiexponential Representation

For ω(j), as classical in polymer physics to account for the excluded-volume effect in the single-strands, a value close to 2 was chosen for α (α=1.95). The corresponding power-law, ((2j+2)^{-1.95}) was numerically represented as a sum of 14 exponential functions, with the help of the Padé-Laplace method (as in FIG. 2D in Yeramian and Clayerie, 1987), with a further refinement by a least-squares fitting procedure.

2.4. Energetic and Thermodynamic Parameters

Helix-coil calculations were performed with a standard set of parameters, as already described (Schaeffer et al., 1989). σ was taken as 1/exp(−ΔG/RT), with ΔG=8000 cal mo⁻¹. For the thermodynamic description of the stacking of bases in the helical state, the 10 dinucleotide stability constants of Gotoh and Tagashira (Gotoh and Tagashira, 1981a; Schaeffer et al., 1989) were used.

2.5. Calculation Times, Accuracy and Robustness of the Results.

For a given sequence, the probabilities in the stability maps were evaluated every 20 base pairs (the partition function taking of course into account all the bases in the sequence). With the SIMEX algorithm, for one temperature, the calculation time (on a Sun Ultra2 workstation, 200 MHz) for the complete probability map associated with the phage lambda sequence (about 48,000 base pairs) was 4 minutes, and 15 minutes for a sequence of length 100,000 base pairs. By comparison, on the same computer, the calculation time for lambda with the exact treatment (in O(n³)) was 5 days. The calculation of probabilities with the accelerated treatment is performed with a numerical accuracy better than 1% (as compared to the probabilities obtained with the exact treatment). Results obtained with the complete model, taking into account the long-range effect, were extremely robust with respect to all (reasonable) choices of parameters. With different sets of parameters, associated with different conditions (ions, pH, etc.), important shifts in the melting temperatures can be observed, with nevertheless essentially no changes in the fine structures of the probability maps. This feature which does not seem to be apparent in the literature will be further discussed in more detail elsewhere.

3.1. Consequences of the Long-Range Effect on the Stability Maps

FIG. 2 illustrates the consequences of including the long-range effect in the model, by comparing stability maps obtained with and without this effect. To do so, we consider the complete sequence of the yeast Saccharomyces cerevisade chromosome VIII (562,638 base pairs; for an overview of this genome see Goffeau et al., 1997). In FIG. 2A, the GC % curve associated with this sequence is plotted with a 500 base pair sliding window. This plot provides only limited information with respect to the properties we are interested in here. The GC % distribution is only one of the ingredients in the detailed physics of DNA stability. In FIG. 2B the stability map associated with the nearest-neighbour model for helix-coil transitions is plotted for the temperature of 63° C. The probability that a given base pair along the sequence (abscissa) is in the coiled state is plotted between 0 and 1 (a probability of 0 corresponding to the base pair in the helical state). In FIG. 2C, the stability map for the same sequence is plotted for the complete model, including the long-range effect, for the same conditions as in 2B. Comparison of FIGS. 2B and 2C shows that inclusion of the long-range loop-entropy factor introduces a very sharp localisation of the helix-opening probability peaks.

A stability map such as the one in FIG. 2C concerns the properties of the regions in which the disruption of the double-helix occurs the most easily (with a relatively low temperature). Such properties could be important in promoters or origins of replications. Here we are interested in the complete genetic maps as related to stability properties. For such a comparison we need more detailed information, beyond the description of easily disrupted regions. More precisely, we need to monitor how DNA stability changes over a range of increasing temperatures. FIG. 2D shows a close-up of a region in yeast chromosome VIII (40,000 base pairs) with the superposition of 5 stability maps corresponding to 5 different temperatures (from 63° C. to 67° C. with different color codes as indicated in the figure). This map displays a fine structure, representative of the whole yeast genome. Maps such as that in FIG. 2D were derived for the complete sequences of all 16 yeast chromosomes (representing a total of roughly 12 million base pairs, in stretches of 100,000 base pairs for chromosomes larger than chromosome VIII). A similar study was performed previously (King, 1993) for chromosome III of this genome with, in this case, the implementation of the complete physical model. Nevertheless, with the representation chosen (a ‘temperature contour for 50% helicity’), the map displayed in this study did not exhibit the fine structure observed in FIG. 2D.

3.2. Superposition of Genetic and Stability Maps

Let us now superpose the genetic maps on the stability maps. First, in FIG. 2E, such a superposition is considered only with the contour of the stability map with a rather high temperature (68° C.). The sequence in this figure corresponds to a close-up of the yeast sequence in FIG. 2D (restricted to a region of 20,000 base pairs). The contour of the stability map with the complete physical model is shown along with the frontiers of 8 genes (YHL003c to YHR006c). As a further demonstration of the importance of the choice of the physical model, the nearest-neighbour map (for the same temperature) is also superposed on this plot. The peaks in the nearest-neighbour model are dispersed quite homogeneously throughout the coding and the non-coding regions. In contrast, a clear-cut discrimination is obtained between the coding and non-coding regions with the complete model. A significant correlation appears between the genes and the stable non-disrupted regions. This correlation is nevertheless imperfect with, for example, certain genes lacking a stability frontier at the end (YBL002w) or at the beginning (YHR006w). Before trying to decipher in more detail the somewhat imperfect correlation observed above for the yeast genome, it is informative to ask what is the result of similar superpositions of the genetic and stability maps in other genomes. The observations in what follows are based on the analysis of the stability maps (such as in FIG. 2D) which were derived for the complete genomes available, as well as for the large stretches of annotated sequences in the databanks.

In FIG. 3, we consider, for two different genomes, the superposition of the genetic annotations on the stability maps. First, in FIG. 3A we consider the map for 20,000 base pairs of the complete mitochondrial genome of Prototheca wickerhamii (55,328 base pairs; Wolff et al., 1994). As seen in this figure, the frontiers obtained with four different temperatures delineate most of the genes as regions in the helical state, in contrast to the intergenic regions, which appear to be in the coiled state with a probability of 1. In such a picture, a gene is a stable region, with a propensity for disruption of the double-helix in the intergenic regions and with a very precise delineation of the frontiers. Cox3 would be a prototypal gene in this respect. As seen for this gene (with its left-end and right-end frontiers), the frontiers that delineate the intergenic regions can be obtained with different temperatures, with nevertheless a significant stability difference between the coding and non-coding regions. The accuracy of the correlation between the genetic and physical stability maps is even better appreciated with the 14 tRNA (ca 80 base pairs) genes in the sequence displayed in FIG. 3A. The frontiers of the genes are very precisely delineated with the exception of 4 tRNA genes (located between positions 25,000 and 26,000 base pairs in the sequence), which are part of a single stability region, devoid of internal frontiers.

In contrast to the previous example, we will now consider 30,000 base pairs of the complete Mycobacterium tuberculosis genome (total length of roughly 4.4 million base pairs; Cole et al., 1998); FIG. 3B. In this case it appears that there is essentially no correspondence between the stability and the genetic maps. With the exception of particular genes not relevant for the general discussion here, the randomly chosen region in FIG. 3B is representative of the whole genome. Also, here we are concerned with the genetic map as describing the delimitation of coding regions. The absence of correlation at this level does not preclude the possible existence of correlations between physical properties of DNA and other genetic signals such as promoters or origins of replication. This matter will be discussed in more detail elsewhere.

These examples are typical of very many sequences studied. It is not possible, nor necessary, to multiply such examples here. The mere existence of such extreme cases is enough to suggest that a presumably perfect correlation between the genetic and stability maps must have existed for all organisms at some remote time, and that it had an important functional role that is no longer relevant. In such a scheme the sharp delineations associated with the differential stability properties of the DNA double-helix might have been used for (or have helped in) an archaic delimitation of coding regions, before the emergence of the modem transcription machineries. Such an hypothesis would account for the observed results, with a progressive erasure of the antique signature occurring at different rates for various genomes. As the signature concerns the delimitation of genes, this hypothesis is compatible with—and can complement—the generally accepted evolutionary scheme following which differential selective pressures operate on various genomic regions, depending on their coding or non-coding nature (Gillespie, 1991, page 84; Osawa et al., 1987). Following this scheme spacer DNA regions are much more susceptible to the ‘GC/AT pressure’ than coding regions, for which constraints concerning the amino-acid sequence of the protein product are involved (with nevertheless some flexibility allowed by the redundancy of the code). Under such light, the erasure mechanism could concern a progressive attenuation of the supposedely primitive sharp delimitation of coding regions provided by the DNA physics, this attenuation being allowed for by the comparatively small selective constraints operating on non-coding regions.

By comparing evolutionarily connected sequences, it should then be possible to trace the effects of the supposed erasure mechanism at various stages. This can be studied by analysing the duplicated genes in the yeast complete genome with respect to stability properties.

3.3 Yeast Genome and Duplicated Genes

In Saccharomyces cerevisiae, the only completely sequenced and annotated eukaryotic genome, a high percentage of genes appear to be duplicated (Lalo et al., 1993; Coissac et al., 1997), as in the other known genomes. A detailed study of the duplicated information revealed about 50 duplication regions (Wolfe and Shields, 1997), defined as series of at least three pairs of homologous genes, between two chromosomes (with intergenic distances of ≦50 kilobases, in each chromosome), with conservation of the order and the orientation of the genes. It is supposed that two duplicated regions originate from a single sequence, with extensive subsequent rearrangements. Such duplicated blocks can thus be appropriate places to look for traces of the erasure mechanisms.

In FIG. 4A, we consider one of the duplicated chromosomal regions (designated as block 9 in Wolfe and Shields, 1997), in chromosome III, with its duplicated counterpart being in chromosome IV. In this figure, the stability map is plotted for the region extending from gene YCL048w to gene YCL035c. With 5 different temperatures, the frontiers for almost all of the genes are clearly delineated. As in FIG. 3A above, the frontiers of the various intergenic regions are obtained for different values of the temperatures. Nevertheless, as seen at the level of the complete yeast genome, the open regions obtained with these 5 temperatures correspond almost exclusively to intergenic regions, delineating the frontiers of a high percentage of genes (one or both extremities). For higher temperatures it is essentially for the bulk of the genic regions that significant opening probabilities are obtained (FIG. 4B). Certain intergenic region frontiers are also only observed at such high temperatures. With respect to stability properties, such intergenic regions with ‘attenuated’ frontiers are barely distinguishable from the genic-regions. This feature is illustrated in FIG. 4B with the intergenic region between YCL037c and YCL036w, whose stability frontiers are observed only at temperatures ranging from 68 to 71 degrees.

We will now focus on two contiguous genes, YCL043c and YCL040w, as part of the duplicated genes in block 9. The duplicated homologues for these genes are respectively YDR518w and YDR516c in chromosome IV (with sequence identities at the amino acid level, of 41% and 73%, respectively). The stability map for the genic region to which these genes belong is displayed in FIG. 4C. In chromosome III, both genes are delineated by stability frontiers whereas in chromosome IV YDR5 1 6c is not delineated by such a frontier at its beginning, and YDR518w is not delineated by stability frontiers at either of its extremities. In fact, with respect to the 5 temperatures defined above, the latter genes belong to a single stability domain beginning with the end of gene YDR516c and ending with the end of gene YDR519w. Compared to their contiguous homologues in chromosome III, YDR516c and YDR518w are separated by another gene (YDR517w). At the high temperature values as defined above, an ‘attenuated’ frontier is observed at the beginning of the gene YDR516c, whereas the end of gene YDR518w is completely integrated into its genic environment (FIG. 4D).

Complete integration of a genic frontier within its environment is referred to here as ‘stability assimilation’ (by analogy with the expression ‘compositional assimilation’ (Li, 1997)): the intergene and the-bulk of the two adjacent genic regions become indistinguishable with respect to stability properties. It is possible that stability assimilation represents the ultimate phase of a progressive erasure, in which the attenuation of stability frontiers is only an intermediate stage. Underlying the assimilation concept there is the hypothesis that such events (as compared to ‘intact’ traces of the archaic signature) must be associated with ‘hotspots’ of chromosomal rearrangements, whatever their precise mechanisms.

Such a hypothesis will have direct consequences for understanding the origin of the observed topography of duplicated regions. Thus, for the genes in block 9 considered above, the rearrangement of stability properties is most visible in chromosome IV and it is natural to suppose that gene YDR517w corresponds to an insertional event rather than a deletion event. With the information provided by the stability maps, it is indeed easier to imagine that the region in chromosome III, with its intact frontiers, is the one whose organisation is closest to the ‘blueprint’ region. It should be noted that in the paper by Wolfe and Shields (1997) it is the deletion hypothesis which that was systematically favored. However, up to now, it was not possible to discriminate unambiguously between the different possibilities that could account for the topography of duplicated regions (extreme divergence between originally duplicated genes, deletional events and insertional events, see for example Coissac et al., 1997). It may well be tha considerations such as those above will ultimately provide the best way of discriminating between different evolutionary events.

Taking this reasoning one step further it should be possible, in many instances, to find ‘original’ copies of the duplicated genes that correspond to the archaic prototype as defined above. It indeed appears that such is the case. As an illustration, we consider again the region [YCL040w-YCL043c] in FIG. 4A. This region was considered to be closer to the original duplicated block than [YDR517w-YDR516c]. However, even in this [YCL040w-YCL043c] region the stability frontier at the beginning of YCL040w does not correspond strictly to the genic frontier. Significantly there exists a homologue of this gene in chromosome VIII (YGL253w, with amino acid sequence identity of 35%) that strictly fulfills the prototypal characteristics of the supposed archaic gene (FIG. 4E). For the validity of the interpretations above it is important to seek more direct evidence for a causal connection between genic rearrangement and stability assimilation. With this respect we shall now examine the scheme in the light of the properties of tandemly duplicated genes.

3.4. Tandemly Duplicated Genes in Yeast

In addition to simple and block duplications, the yeast genome, includes examples of tandem duplications. In FIG. 5A we consider two such examples as direct probes for the effect of localized rearrangements on physical stability. In FIG. 5A we consider the tandem duplication of the three genes [ENA1-ENA2-ENA3] in chromosome IV (respectively YDR038c, YDR039c and YDR040c). With amino acid sequence identity scores ranging between 98% and 99%, this tandem duplication involves minimal genic rearrangements. As seen in the figure, all the genes are strictly delineated by sharp stability frontiers. Another tandem duplication concerning a family of genes in chromosome I (YAR027w to YAR033w, with the exception of the gene YAR030c) is shown in FIG. 5B. With sequence identity scores (at the amino acid level) between 14% and 58%, the tandem duplication must have involved significant rearrangements together with the probable insertion of the gene YAR031w. All of the intermediate stability frontiers between the genes are erased, leading to a single homostable region. At the high temperatures, the erasure of frontiers is associated with a complete stability assimilation (as defined above) for all the duplicated genes within the local genic environment (FIG. 5C).

The deciphering of the information derived from the superposition of three types of data (genetic and stability maps together with duplication information), can be termed ‘paleogenomics’. The evidence for the existence of a signature of what must have been part of an archaic genetic system raises many evolutionary questions. In such a context, the fundamental physical difference between AT and GC base pairs can appear as an operative feature, selected for by evolution, as the basis of sharp delineations between coding and non-coding regions. As the archaic organization—whatever it's origin— became obsolete, the erasure of the signature could either be a consequence (for features no longer under selective pressure), or one of the causes at the origin of evolutionary drifts.

The complete dynamics of genome evolutions (leading, for example, to GC contents ranging from 15% to 80%) should be considered in such a light, with, notably, the possibility of non-random mutations as part of an effective erasure mechanism. One intriguing possibility is that the erasure mechanism with the underlying stability assimilation could have played a direct role in the observed striking variation in GC composition. It is noteworthy that the above selected examples span the range of possible GC % (about 26% in the Prototheca wickerhamii genome, about 38% in the Saccharomyces cerevisiae genome and about 66% in the Mycobacterium tuberculosis genome). The various examples were discussed here individually, for their own sake, irrespective of such differences in composition. Such examples may reflect an overall correlation, though unspecified evolutionary trends, between a distinctive thermal stability for coding sequences and the global GC composition of given organisms. In any case, this trend is not the only one involved, as proved by the existence of extremely AT-rich sequences for which the thermal stability feature of coding regions does not hold. More speculative treatments providing possible schemes that could explain the observed divergences for various organism will be presented elsewhere. Also, for simplicity, all the treatments here were limited to uninterrupted genes. Of course the question of the correspondence between physics and genes may be raised at the level of split genes as well. Some aspects of this question are dealt with in the companion paper (Yeramian, 2000), which is essentially devoted to the potential practical usefulness of the structural analyses in terms of gene identification and genome annotation.

Beyond the helix-coil model, large-scale structural formulations can be extended to other models as well. The treatments here can provide a necessary foundation for such large-scale structural studies, notably with respect to the methodological and algorithmic problems involved in the implementation of long-range effects.

In summary, the processing of the genetic information stored in the double-helical DNA implies the separation of the two strands, the physics of which is described by the helix-coil transition, model. Is there a relationship between genetic maps and DNA physical stability maps, which plot the sequence-specific propensity for the thermal disruption of the double-helix? Here, with appropriate methodological formulations, such maps are derived for a large set of sequences, including complete genomes. The superposition of the two maps leads to a contoured picture with correlations ranging between two extremes: from almost perfect (with the genes precisely delineated as stable regions) to more or less complete unrelatedness. The simplest explanation for the results is that the observed striking correlations correspond to the relics of a primeval organisation of the genetic message, with the physics of DNA playing a role in the delimitation of coding regions. In order to trace the evolutionary fate of this signal further, a detailed study of the yeast complete genome is performed. In this study, the superposition of the genetic and physical stability maps is examined in the light of information concerning gene duplication. On the basis of this analysis it is concluded that the ‘signature’ associated with the supposed archaic signal is in the process of being erased, most probably because the underlying feature is no longer under selective. pressure. There are many evolutionary implications for the results presented and for their proposed interpretations, notably concerning models of mutational dynamics in relation to erasure processes.

In another embodiment of the invention, an ab initio gene identification procedure is formulated, based on large-scale structural analyses of genomic sequences. The structural property is the physical—thermal—stability of the DNA double-helix, as described by the classical helix-coil model. The analyses are detailed for the Plasmodium falciparum genome and extended to a series of other eukaryotic genomes (Homo sapiens, Anopheles gambiae, etc.). The plasmodium falciparum genome, which represents one of the most difficult cases for the gene-identification problem (notably because of the extreme AT-richness of the genome). In this genome, the coding domains (either uninterrupted genes or exons in split genes) are accurately identified as regions of high thermal stability. The conclusion is based on the study of the available cloned genes, of which 17 examples are described in detail. These examples demonstrate that the physical criterion is valid for the detection of coding regions whose lengths extend from a few base pairs up to several thousand base pairs. Accordingly, the structural analyses can provide a powerful and convenient tool for the identification of complex genes in the P. falciparum genome. The limits of such a scheme are discussed. The gene identification procedure is applied to the completely sequenced chromosomes (2 and 3), and the results are compared with the database annotations. The structural analyses suggest more or less extensive revision to the annotations and also allow new putative genes to be identified in the chromosome sequences. Several examples of such new genes are described in detail.

With the ever increasing availability of genome sequences, the retrieval of the relevant information from the raw data is becoming a critical limiting step in genomic projects. Currently, gene identification relies either on sequence homologies or on predictions, most often of the pattern recognition type. In pattern recognition methods, a biological sequence is assimilated to a text made of a limited number of characters, in which the appropriate signals must be discovered. Most often such methods involve training steps, relying on neural networks or hidden markov chains (for a general overview see for example Baldi and Brunak, 1998, and references therein). As such, the predictive tools with the trained sets are best when they encounter situations similar to those with which they were made ‘familiar’. These various approaches have proved successful and very helpful in many cases, but their limitations are now obvious. New—alternative—gene identification methods are becoming necessary, notably for the most difficult cases. One possible approach is the development of identification procedures based on structural models, as structural signals play important roles in various steps of the processing of the genetic information. Such approaches would bring back ‘flesh and bones’ to a purely textual reading of the biological sequences, in which the underlying physical templates are essentially ignored.

The object of the present work is to formulate a gene identification procedure based on large-scale structural analyses of genomic sequences. The structural property used here is the thermal stability of the DNA double-helix, associated with the sequence-specific propensity for the opening of the DNA double-helix under the effect of temperature (or any other denaturing agent). This property is described by the classical helix-coil model, which is detailed in an extremely large literature (notably in a series of textbooks, see, for example, Poland and Scheraga, 1970, and Cantor and-Schimmel, 1980). The various methodological issues (physics, algorithms) for the large-scale implementation of such a model, at the level of complete genomes or chromosomes, are discussed in the companion paper (Yeramian, 2000, this issue). Here we shall examine the potential practical usefulness of structural approaches in the gene identification problem.

According to the results detailed above, the prospects for a physics-based gene identification procedure could appear as rather mitigated. While this work demonstrated the existence of a distinct relation between genetic maps and physical stability maps (which plot the probability of opening of the double-helix along the sequence), the levels of correspondence clearly varied between the genomes and the organisms. The conclusion of this work is that the result should be examined in an evolutionary perspective. Following this perspective, it should not be expected that a procedure such as the one considered here could be applied with the same effectiveness in all cases.

The example we shall consider concerns the genome of Plasmodium falciparum (responsible for malaria). At present two chromosomes of this genome have been completely sequenced (the chromosome 2 by Gardner et al., 1998, and the chromosome 3 by Bowman et al., 1999). This genome is supposed to represent one of the most difficult cases for the more traditional gene identification methods. The P. falciparum genome is—atypically AT-rich (GC content around 20%), and displays rather complex genes, which can consist notably of series of small exons. It should be noted that in the results reported above, simple (uninterrupted) genes are considered, without the additional complexities attached to split genes.

In order to gain a general idea for the structural description of split genes in P. falciparum, we consider first a series of snapshots from one of the completely sequenced and annotated chromosomes (chromosome 2). These snapshots show that the superposition of the physical stability maps and the genetic maps (concerning simple as well as split genes) leads to contrasting results: almost perfect correspondences in certain cases and partial or total divergences in others. When perfect correspondences are observed, coding domains (either simple genes, or exons in split genes) appear as regions of high thermal stability with sharp delimitations of their frontiers.

The genes in the chromosome annotations correspond to predictions. In order to assess the adequacy of the physics scheme for predicting coding domains we need, accordingly, to turn to cloned genes. A detailed analysis of cloned genes is performed, based on a set of 17 examples (simple genes as well as split genes). This set represents, notably for the split genes, a significant portion of the available cloned genes in the databases. The described set also provides a rather large spectrum for the possible complexities of the genes, relative both to the lengths of the coding regions (from a few base pairs up to several thousands base pairs) and to the number of exons (in the case of split genes). This benchmark reveals a striking correspondence between the structural properties and the annotation of the cloned genes. Even if the correspondence is not always perfect, it appears that the type of discrepancies in the cloned genes could not account for the divergences (when they are observed) in the analysis of genomic sequences. The discrepancies in the cloned genes essentially concern the presence, in certain cases, of low-stability regions within large open reading frames. Such discrepancies need to be treated as special cases. For, the predictive purposes, it is possible to let aside such cases, based on the ORF-analyses.

With the study of the cloned genes, it appears possible to elaborate a physics-based scheme for the identification of genes in P. falciparum. In such a scheme, we are led to suppose that annotations which contradict the physics should correspond to more or less serious errors in the predictions. Such a scheme may be applied to the completely sequenced chromosomes, proposing rectifications to the annotations whenever they are contradicted by the physics. In the same logic, we can also proceed to the identification of new putative genes when the physics reveals the presence of coding regions not reported in the annotations. Based on the physical information, complete gene identification involves, of course, further detailed exon-assembly (identification of splice signals and ORF-analyses). Such a complete procedure is illustrated with several examples.

All treatments are as described above. We only recall here that we need to evaluate, for different temperatures, the stability maps along given sequences. By definition, for a given temperature, a stability map plots the probability for a base pair to be in the coiled state (with the probability zero being associated with the helical state, and the probability one with the fully open—coiled—state for the considered base pair). The probability curves are evaluated with the SIMEX method (Yeramian et al, 1990; Yeramian, 1994). In order to make the calculations tractable, the long-range effect in the model (associated with the physical representation of the denatured loops) is numerically represented as a sum of exponential functions, by use of the Padé-Laplace method (Yeramian and Clayerie, 1987).

Concerning the thermodynamic parameters (see above) the important consideration here is that the results appear to be very largely independent of the choice of any—reasonable—set. In fact, for the probability curves as displayed here, the same results (with marginal variations) could be obtained even with the less sophisticated sets of parameters. This feature was checked notably with the parameters which were in use in the late sixties and early seventies (only two equilibrium constants—associated with the AT and GC base pairs—with a constant proportionality of 5.3 between them, see for example Crothers and Kallenbach, 1966).

All calculations are performed with the evaluation of the probabilities every 20 base pairs (unless otherwise specified). Every probability calculation does however take into account the complete sequence. In the stability plots, the curves are drawn with graphical interpolations for the base pairs for which the probability calculations are not performed. Unless otherwise specified, in each figure a set of 5 temperatures is used (56° C., 57° C., 58° C., 59° C. and 60° C. denoted as T56 to T60 respectively). This set is chosen empirically, for the P. falciparum sequences. Of course this temperature set is relative to the conditions (pH, salt, etc) associated with the thermodynamic parameters used. For other thermodynamic parameters shifts in the temperatures used in the analyses can occur, without affecting the differential descriptions associated with the probability curves. Different color codes are associated with the 5 chosen temperatures, as indicated in FIG. 6. Unless otherwise specified these conventions are used throughout the paper.

For the complete chromosome sequences the calculations (unless otherwise specified) are performed by stretches of 100,000 base pairs. In the corresponding figures, the origin is set at the first base pair of a given stretch. For the two complete chromosomes (2 and 3) we adopt the following conventions: a notation such as chr2(100_{—}200) designates the region in chromosome 2 spanning the sequence from 100 kbp to 200 kbp.

All database annotations (with the corresponding accession numbers) are from the NCBI web site. Sequence analyses for the determination of open reading frames (and the further assembly of exons into genes) are performed with the ‘DNA Strider’ program (Marck, 1988). For the comparison between physical stability maps and genetic maps, experimentally determined or putative coding regions (simple genes or split genes) are indicated as colored horizontal bars with vertical arrows at their beginnings and their ends, in strict correspondence with the beginnings and ends of the considered coding regions.

3.1. Snapshots from the Chromosome 2

In FIG. 6 we consider 3 snapshots for stability curves, from 3 different regions of chromosome 2. As mentioned above all stability curves plot the probability for coiled regions—for given temperatures—along the sequences. On the same curves, the genes corresponding to the database annotations are reported. In this figure the GC % of the corresponding regions are also plotted along the sequences, allowing a detailed comparison between the different types of information.

FIG. 6A reveals a striking correspondence between the stability curves and the genomic annotation, for the 5 genes in this region (their names are indicated above the colored bars). The conclusion holds for the simple (PFB080c) and split (PFB0075c, PFB0085c, PFB0090c, PFB0095c) genes as well, independently of the length of the coding regions. In this correspondence, exons in split genes are described in exactly the same terms as simple genes, relative to the physical stability properties. For simple genes as well as for exons, coding regions appear as clear-cut domains of high thermal stability compared to intergenes or introns (non-distinguishable per se at this level), with sharp delimitations of the frontiers. Of course, the GC % follows the same general trends as the complete physical stability description. However, it would be more difficult using the GC % information alone to discriminate clearly between the coding and non-coding regions. This feature becomes all the more striking with the increasing complexity of the examples considered.

At the level of FIG. 6A the only notable discrepancy between the database annotation and the analysis based on the physical stability maps concerns the second gene (PFB080c), for which the physics suggests the presence of an additional small exon at its right-end (corresponding to the high thermal-stability spot).

FIG. 6B also shows a rather sharp correspondence between the physics and the annotation. This holds true notably for the first-half of the sequence. In this first-half the discrepancies may correspond to the presence of a few small additional coding regions, as suggested by the physics (for example between the genes PFB0295w and PFB0300c). In the second-half of the sequence the situation is rather different with the discrepancies being relevant to two low-stability regions observed within a large open reading frame (gene PFB0315w).

As a last example we consider the sequence in FIG. 6C. In this figure we can observe the coexistence of the various possibilities (between two extremes) for the correspondence between the physics and the database annotation: 1) a rather perfect correspondence for the first split gene (PFB0355c, a close-up view of which is displayed in FIG. 6D, left panel) and the last simple gene (PFB0370c); 2) a good correspondence for the second gene (PFB0360c), with the exception of a low-stability region within the first exon and the absence of a low-stability region between the first and the second exons, and, finally 3) a rather poor correspondence for the gene PFB0365w for which the right extremity corresponds to a sharp frontier for the physics, but such a frontier is lacking at the left extremity (in addition, we also observe low-stability regions within this gene). If we consider this last gene (PFB0365w) within its genomic environment, we can observe at its left-side a large number of small high-stability peaks (or alternatively a large number of small low-stability spots), which might indicate the presence of a series of small exons (either to be assembled with the gene PFB0365w, or constituting a new separate gene). For this region associated with putative small exons, a close-up view is displayed in FIG. 6D (right panel).

This snapshot overview gives the general impression that a striking correlation can exist between the genetic and physical stability maps, for both simple and split genes. This correspondence is nevertheless not perfect, as discrepancies of several types exist from small differences in the two representations up to complete non-correspondence. It is then tempting to try to determine to which extent the physics-genetics correspondence should be considered as contriving in the case of the P. falciparum genome, with predictive virtues for the genomic annotations. The only possibility in such a direction is to submit the physics-based scheme to the benchmark of known (cloned) genes.

3.2. Analysis of-Cloned Genes. Validation and Calibration of the Physics-Based Gene Prediction Scheme

We consider here a set of 17 cloned genes as found in the databases and for which the annotations correspond—in principle—to experimentally confirmed data. Details concerning the various genes are provided in the legends of the figures.

3.2.1 Simple Genes

In FIGS. 7A-D we consider four simple genes. In these four examples the correspondence between the frontiers of the cloned genes and the physics appears to be close to perfect, irrespective of the length of the genes (up to roughly 9000 base pairs). One important point to note in these examples is that insert sequences do not appear to be associated with spurious signals in the stability curves. Such inserts, occurring rather frequently in P. falciparum, concern the presence within a gene of sequences not observed in families of similar genes in other organisms. This observation is illustrated for example by the gamma-glutamylcysteine synthetase gene (FIG. 7A), characterized by a series of large inserts.

3.2.2 Split Genes

FIGS. 7E-M show the analysis of nine split genes. In FIGS. 7E-I there is a close correspondence between the exons in genes and the thermal high-stability domains. It can be noted that different temperatures can be associated with the helix-opening of the various introns (for example in 7F the opening of the first intron corresponds essentially to the temperatures T58 and T59, whereas the opening of the second intron corresponds to the temperatures T56 and T57). These examples also show that the physics-genetics correspondence holds for small as well as large exons (such as the first exon in 7G, roughly 6500 base pairs long).

The examples in FIGS. 7J-M correspond to more complex cases, which will be analyzed in some detail. The example in FIG. 7J corresponds to a gene with 4 exons. The interesting point in this example is that the first exon (33 base pairs) is significantly smaller than the other ones. Unlike the other exons, there is no stability ‘cuvette’ or ‘sink’ associated with this exon. Instead, the exon is harbored in a stability domain characterized by a ‘shoulder’ structure for the T59 (not saturating to the value 1) and T60 curves. The shoulder structure can be described as resulting from the merging (in terms of stability properties) of the exon 1 to the intron-domain separating it from the exon 2. Indeed the right-end frontier of the shoulder structure strictly corresponds to the left-end frontier of the exon 2, whereas the right-end frontier of the exon 1 corresponds to no stability frontier within the shoulder region.

To calibrate the method as a predictive tool, such configurations must be taken into account properly. In what follows we shall call EHSR (‘Exon Harboring Stability Region’) a clearly delineated domain, characterized by homogeneous stability properties, inside which exons can be found with—at least—one of their frontiers not fixed unambiguously by the physics. The shoulder structure as described above corresponds to one such configuration for an EHSR.

In this example, anecdotally, at the very beginning of the sequence the physics displays a high-stability region, which appears to coincide exactly with an open reading frame.

The gene in FIG. 7K provides another example for the occurrence of such an EHSR, with a shoulder structure for the T59 and T60 curves. The EHSR corresponds to the stability domain which harbors the last exon (5). The left-end frontier of this exon is not delineated by physics stability. In a configuration similar to the one above, the left-end frontier of the shoulder region corresponds to the right-end frontier of the previous exon (4). Another interesting observation in this example concerns the first intron (between exons 1 and 2), the frontiers of which are strictly delineated by the T60 curve. However, this intron appears to be rather atypical compared to the cases above, with an ‘attenuation’ of the helix-opening conditions. The attenuation concerns both the temperature (frontiers seen only with T60) and the height of the peak (the T60 curve not saturating to the value 1).

FIG. 7L displays a gene made of 9 exons, for which alternative splicing has been demonstrated (see the legend). As seen in the figure, with the usual conditions as used throughout this paper, all exons are properly detected by the physics stability scheme with two exceptions: there are no intermediate stability frontiers between the exons, 1 and 2, and the exons 3 and 4 (respectively). In each case, the exons are harbored in a homogeneous stability cuvette, which can be assimilated to a domain of the EHSR type. In contrast to the shoulder structure above (stability of an exon homogeneous with that of a contiguous intron), in the cuvette structure it is the stability of an intron which is homogeneous with that of the two contiguous exons. The intron between exons 5 and 6 is revealed by the T60 curve (this intron being of the ‘attenuated’ type as in the previous example, with an even smaller height of the associated peak). In a configuration such as this we can ask whether the introns missed by the physics might be recovered with still higher temperatures. Accordingly, in the figure, two additional stability maps corresponding to the temperatures T61 and T62 are further reported (as red lines). It appears that such additional information could sometimes be useful to increase the resolution of the analysis. For the intron-separation between the exons 3 and 4 a small stability ‘hill’ (in red) appears with the new curves. With the higher temperatures a different kind of—possible—analysis enhancement can be also observed: certain exons are delineated by ‘depressions’ seen in the T61 and T62 curves. Such an effect can be observed notably for the exon 1, but also for the exons 5, 7 and 9 (already detected with the standard conditions). In certain difficult cases it could be useful to resort to such more detailed analyses, using the corresponding information as confirmations or exploratory hints. In any case, the physics analyses must be ultimately complemented with classical sequence-analyses, concerning the determination of open reading frames. Signals not detected with the physics analyses should often be readily resolved at such a step. For example in the gene considered here, in the absence of cloning information, the physics would fix immediately the overall limits of the stability, region which harbors the first two exons. This information signals immediately (with ORF-determinations) that this region cannot correspond to a single exon (no matter the reading frames). Taken together, the two informations allow one to proceed (easily) to a correct exon assembly. It is possible however that in certain cases, when some physics frontiers are lacking, more than one solution may exist.

In FIG. 7M we consider a gene with 16 exons, in a 3.8 kbp sequence. Even in this rather complex example there is a rather striking agreement between the physics stability analysis (with the routine conditions) and the annotation of the cloned gene. Interestingly, the possible discrepancies appear to correspond again to some lacking intron-frontiers, but with otherwise no spurious signals. The last part of the gene (exons 13 to 16) can be described as the juxtaposition of the two types of EHSR structures already seen above: a cuvette structure (exons 13 to 15, with both stability frontiers lacking for the exon 14) immediately adjacent to a shoulder structure (harboring the exon 16). The left-end frontier of the shoulder corresponds to the right-end frontier of the last exon (15) in the cuvette. In this case, for the homogeneously stable cuvette region (exons 13 to 15), the T60 curve does not fall to zero. Exons 5 and 6 are also in an EHSR, of the cuvette type. Another observation concerns the exons 2 and 8, for which the high-stability domain are larger than the lengths of the exons. As for the previous example we shall examine potential enhancements of resolution in the physics-analysis. In this example we shall explore two further different variants to the routine conditions. First, in FIG. 2M we consider the possibility of evaluating the stability maps with smaller steps for the temperature increase (0.1 ° C. instead of 1° C.). More precisely, we consider 5 additional temperatures (T59.5 to T59.9, in cyan lines), incremented by steps of 0.1° C. As seen in the figure this additional information does not increase in any significant way the resolution of the analysis. For introns such as the ones between exons 4 and 5, or exons 9 and 10, the additional information only highlights graphically the detection performed with the standard conditions. On the other hand, the fluctuations in the additional curves appear to delineate somehow the frontiers of certain exons (such as for the exon 15). Another potential possibility to enhance resolution might be to avoid interpolation in the stability curves, by evaluating probabilities every base pair (instead of every 20 base pairs in routine conditions). For one particular temperature (T59.7) a comparison between the two types of calculations is displayed in FIG. 7N, showing little difference between them.

3.2.3 Low-Stability Regions in Large Open Reading Frames

In this section we consider a series of examples of cloned genes (FIGS. 7O-R), for which discrepancies exist between the genomic annotation and the physical stability maps. In each case the discrepancy is relevant to the observation of low-stability regions within large open reading frames.

The gene in FIG. 7O is reported as a. simple gene. Nevertheless we can observe in this gene a low stability region, which would be assigned as an intron in a strict application of our physical scheme. As a matter of fact an ORF-analysis can be performed for this sequence with the low stability region assimilated to an intron (with the proper consensus intron/exon splice sites, see legend). We cannot resolve completely this discrepancy at this stage. It is however interesting to notice that stage-specific expression was demonstrated for this gene with two major bands of 80 and 90 kDa, recognized by anti-pepB serum in Western blots (Mohrle et al., 1997). The ratio of the length of the coding region obtained from the physics analysis with the low-stability region removed, to the total length of the coding region, gives the figure 0.9 which is quite close to the ratio 80/90. While no definitive conclusion can be drawn from this simple observation, it nevertheless suggests that perhaps we should not dismiss too quickly the detailed examination of such discrepancies as they may be related with some potential interesting functional features, at least in certain cases.

The intron in the split gene in FIG. 7P is correctly depicted by the physics, as a sharp region of low-stability. In addition, in the second large exon, we can observe a series of small signals (of the ‘attenuated’ type, following the terminology above) associated with the T60 curve. Similarly, in the split gene in FIG. 7Q, we can observe (in the large first exon) a series of signals of the same type as in the previous example. For the split gene in FIG. 7R we observe a rather complex structure for the discrepancy between the physics and the reported annotation (5 exons). For the last three exons (3, 4 and 5) the correspondence is almost perfect. We observe one sharp low-stability peak in the second exon. Within the first very large exon we observe three low-stability regions of the attenuated type and, in addition, two low-stability peaks corresponding to lower temperatures (T56 to T58). On the basis of the analysis of the paper describing the cloning of this gene (Dr. Bonnefoy, private communication) it appears that for this large coding region detailed RT-PCR experiments were not performed. Nevertheless, sequence-comparison results seem not to favor the presence of genuine additional introns, in this region (Dr. Bonnefoy, private communication).

To conclude this chapter it is interesting to put in connection the observation of low-stability regions in large open reading frames with some other features, which can be either trivial or will deserve more detailed elucidations. First, as already mentioned above, in P. falciparum genes it is rather frequent to observe large insert sequences. For example, large (kinase) inserts are observed in the PfPK4 gene (FIG. 7O). As for the gene in FIG. 7B these inserts are not associated with low-stability regions. Second, an unusual abundance of nonglobular domains in P. falciparum proteins has been reported (Gardner et al., 1998, Bowman et al., 1999). These domains (which do not assume compact structures) are essentially determined on the basis of compositional complexity in the amino acid sequences (SEG program; see for example Wootton and Federhen, 1993). It does not seem that a trivial correspondence exists between such domains and the observed low-stability regions. For example, the large nonglobular insert in the gene PFB0180w (discussed in detail in Gardner et al., 1998) is not associated with any low-stability region (result not shown). On the other hand, low-stability domains can be rather trivially associated with certain sequences displaying low-complexity at the DNA level (such as A-tracts).

3.2.4 Blind-Tests

In this last section, on the testing and the validation of the physics-based scheme, it is interesting to mention two blind-tests performed recently in collaboration with two laboratories (Dr. Bonnefoy and Dr. Fidock). In both cases the physics alone (without any additional information, even not the determination of open reading frames) allowed an accurate determination of the genetic organization of the split genes (with, in one case, a gene complexity comparable to that of the example 7M above).

In conclusion, we can consider the application of the physics-based scheme as a predictive tool for the identification and annotation of genes in the P. falciparum genome. The standard conditions used here (the 5 temperatures from T56 to T60, and the calculation of probabilities every 20 base pairs) provide the appropriate informations in most cases. It also appears necessary to examine in detail the EHSR regions, as defined above.

3.3. Physics-Based Analysis of the Genetic Message in Chromosomes 2 and 3

With the validation, and ‘calibration’, of the predictive tool we return to the analysis of the two complete chromosomes. Instead of contiguous genes as in the snapshot approach, we consider the genes here following the degree of correspondence between the physics and the database annotations. Following this classification we introduce increasingly more extensive rectifications in the annotations and, ultimately, proceed to the identification. of new putative genes.

3.3.1 Identification of Genes with Known Similarities

In FIG. 6 we observed for a series of genes a good (or close to perfect) correspondence between the physics and the annotations. A closer inspection of the examples in this figure reveals that in most cases the corresponding genes are identified by sequence singularity (in the database annotations). This is the case for genes PFB0085c to PFB095c in FIG. 6 for genes PFB0290c to PFa.0310c in FIG. 6B and for the three genes PFB0355c, PFB0360c and PFB0370c in FIG. 6C. For the other genes the annotations are based on the Glimmer prediction method (Salzberg, 1998). For certain genes in this last category a good correspondence is observed (for example, for the rather simple genes PFB0075c and PFB0080c), but such is not the case for the gene PFB0365w.

The observation on the genes predicted by similarity appears to be rather general at the level of the complete chromosomes. For example in chromosome 2 there are 208 genes in the database annotation, out of which 116 are reported as identified by sequence similarity. With possibly one exception, for all these genes a good correspondence between the physics and the annotation is observed. For this category of genes, possible discrepancies are of two types: a) in 16 genes, low stability regions are observed within certain open reading frames, and, b) for a series of genes the physics suggests rectifications and corrections to the annotations (such as appending new exons to given genes). The suggested rectifications are of variable extent, but do not disrupt drastically the overall basic structures of the genes. Such possible rectifications are illustrated by a series of examples (FIGS. 8A-F) from both chromosomes (for the chromosome 3, following the available annotation, it appears to be more difficult to determine precisely the number of genes identified by similarity).

The gene in FIG. 8A (with 4 exons) is a representative example where the annotation and the physics are in complete agreement. Such is also the case for the two genes in FIG. 8B. However, the interesting point in this example is the identification of a small, high-stability spot between the two genes. An ORF-analysis shows that we earl associate a very small exon with this stability spot (coordinates in the legend), appended to the second gene (in red). In the figure this additional exon is represented in green. We shall use this color-convention for all the proposed rectifications and new annotations. It can be pointed out that isolated exons such as the one in this example would be difficult to predict with classical methods, but are immediately obvious with the stability curves.

In FIG. 8C we consider a more complex gene. The basic structure of this gene predicted by similarity agrees essentially with the physics. The physics suggests however two rectifications, which concern the exons 4 and 8. For the exon 4 the rectification leads to a shortening of its length from the right-end frontier. For the exon 8 the right-end frontier also is contradicted by the physics (it overlaps a T58 stability domain). If we take into account the ORF analyses, we are led in fact to a rather extensive shortening of this exon from both ends.

As well as suggesting the shortening of coding domains, the physics can also lead to the extension of certain genes. Such is the case, for example, for the gene in FIG. 8D which is extended from 2 exons (1 and 2, in blue) to 6 exons (a to f, in green). The extension appears however not to contradict the basic structure of the portion of the gone predicted by similarity. This is true notably for the intron separating the two exons (1 and 2), in the original annotation. The exon 1 is slightly shortened on its left-side, so that the frontier of the shortened exon (d) corresponds to the right-end frontier of the small T60 hill seen at the left-side of this exon. In such an analysis the T60 hill is assimilated to an intron. It is indeed immediately visible, with the high stability domains at the left-side of the exon 1, that the coding regions should extend beyond this exon. ORF-analysis shows that the right-end frontier of the exon c cannot coincide with the left-end. frontier of the T60 hill (between exons c and d). It can be noticed that, in this case, an appropriate representation of the expected intron (separating c and d) is recovered with the T61 curve (represented in red). At the left-end frontier of the exon c we can observe a sharp low-stability region, with a shoulder structure for the T60 curve (and for the T6 1 curve as well). It appears that, in this case, the shoulder region does not harbor any additional exon. Still on the left-side direction, there is another high-stability domain, corresponding to an EHSR of the cuvette type (harboring two small exons a an b). On the right-side of the exon 2, the physics reveals the presence of a small exon (f).

Of course such rectifications are only putative. They are suggested by the self-coherence of the predictive scheme, as validated with the cloned genes. A more direct argument for the validity of the rectifications can be obtained nevertheless from the similarity analyses themselves. The gene in FIG. 8D was originally identified by similarity with the yeast gene ‘YMR7’, and the score for the similarity analysis (Blast program) is enhanced with the new annotation.

The example in the FIG. 8E (15 exons in a 5.6 kbp sequence) corresponds to a rather complex predicted gene. The overall structure of the similarity-based prediction corresponds well with the physics. The most notable rectification suggested by the physics concerns the exon 1, whose right-end frontier falls in the middle of a high-stability domain. In addition, this exon overlaps a shoulder structure in the T60 curve. Following these observations, and based on ORF-analyses, we are led to replace the exon 1 by the three exons a to c. The new analysis is coherent with the EHSR configurations of the epaulement type, as described with the cloned genes: the exon a is harbored in the stability domain corresponding to the shoulder, and the right-end frontier of the shoulder structure coincides with the left-end frontier of the next exon (b). In the figure, stability plots associated with temperatures T59. 1 to T60.1 (by steps of 0.1° C.) are represented as magenta lines, in addition to the standard conditions. These curves essentially highlight, graphically, the conclusions derived from the standard conditions. For example, it is interesting to inspect in detail the fine structure of the stability curves at the level of the rather large shoulder structure harboring exon a. In the close-up view of this region (FIG. 8F, left), we can observe that the exon corresponds to a ‘plateau’ value for the stability curves which define the epaulement structure. The physics suggests a slight shortening of the exon 2 (leading to exon d). We can also observe two EHSR regions of the cuvette type (harboring exons 3-4, and 8-9, respectively). Another interesting observation concerns the presence in this gene of a second rather large shoulder structure (T60 curve, highlighted by the magenta curves) containing two exons (5 and 6). As usual, the right-end frontier of the shoulder structure corresponds to the left-end frontier of the next exon (7). The close-up view of this region (FIG. 8F, right) reveals however a very small hill in the T59 curve between the exons 6 and 7. If more accentuated, the stability hill would clearly define the intron between exons 6 and 7, and, in such a case, the shoulder structure would be assimilated to an EHSR of the cuvette type (at least relative to the temperatures T56 to T59). This observation suggests that the various types of EHSR structures should be considered as convenient representations, rather than as absolute categories. The close-up view in FIG. 8F (right) reveals that the stability curves associated with the shoulder structure display some fine structures, which bear again correspondence with the frontiers of the exons (5 and 6). Even though the amplitudes of the corresponding effects can be quite small, we observe a plateau value associated with the exon 5 (sharp deflections in the stability curves at the left-side of the exon and very small deflections at the right-side) and a partial representation of the exon 6 (small deflections at the right-side). It is not meant, at present, to draw general conclusions from such observations concerning plateau values in the curves in FIG. 8F (left and right). The observations suggest however that it may be worth examining in detail the fine structure of the stability curves associated with large shoulder regions, in the corresponding EHSR configurations.

3.3.2 Identification of New Putative Genes

For genes predicted without similarity informations, the rectifications suggested by the physics can be even more extensive than in the previous examples. In such cases, the physics can contradict the basic organization of the predicted genes (overall extension of the gene and exon-intron structures). Such an example was already mentioned in FIG. 6C, with the gene PFB0365w. Instead of rectifying such predictions, we shall explore here the possibility of detecting new putative genes in regions where the physics displays signals such as those seen at the left of the gene PFB0365w (FIG. 6C). In all the analyses classical intron/exon splice site consensus sequences are used (see, for example, Table III in Knapp et al., 1991).

In FIGS. 9A to 9F we consider a series of potential new genes in chromosome 2. Completing the physics analysis, an exon-assembly is performed in each case, based on ORF-analysis and the recognition of the proper splice sites. The coordinates for the new predicted coding regions are given in the legend of the figure.

The gene in FIG. 9B is predicted with 5 exons. As for one of the cases above, this figure shows the juxtaposition of two EHSR structures, one of the cuvette type (exons c and d) and one of the shoulder type (exon e). The shoulder, but also the intron between b and c, are only drawn by the T60 curve. As a matter of fact, we can see in the cuvette structure a very small hill drawn by the T60 curve corresponding to the intron between c and d. In FIG. 9C, the same predicted gene is represented with some additional conditions (no interpolations and intermediate temperatures up to T61, see legend). Concerning interpolation, as observed already above, the comparison between C and B reveals the sharpening of some frontiers (for example for the intron between exons b and c). The additional conditions highlight the conclusions in B: notably the intron between c and d is highlighted by the upwards deflections in the stability curves.

The gene in FIG. 9D provides yet another illustration for an EHSR structure of the shoulder type (exons e and f), with, interestingly, small hills in the T59 curve drawing the appropriate introns within this structure (between exons e and f, but also between exons d and e). For this gene the detailed exon-assembly (at the sequence-level) is displayed in FIG. 11, and open reading frame analysis (DNA Strider program) for the assembled gene is represented in FIG. 9D, below the stability plots. The result of database (Blast) search for the new gene is also shown in this figure, with the alignments revealing homology with RING zinc finger proteins.

Similarly, for the gene in FIG. 9E the detailed exon-assembly (at the sequence-level) is displayed in FIG. 12. The analysis in FIG. 9F leads to a gene with 10 exons.

It is of course not possible, nor the aim, to reannotate here both chromosomes in their entirety. To complete this overview for the detection of new genes we shall further consider a series of examples from chromosome 3 (FIG. 10). These examples are also interesting for comparative purposes, relative to the gene detection methods. Exactly one month after the original submission of the manuscript corresponding to this work, two ‘brief communications’ were published in the Nature journal (Pertea et al., 2000; Lawson et al., 2000) discussing the ‘missed’ genes in chromosome 3, as detected by the (Glimmer) method used for the annotation of the chromosome 2. The three examples in FIG. 10 (with the details of the annotations added in the legend) can be compared to the corresponding analyses in the communication by Pertea et al. Based on the physics analysis, the new gene (PFC0585w) in FIG. 10A is composed of 14 exons (with a coding sequence of length 1011 base pairs). This gene can be put in correspondence with the two genes G408 (213 base pairs) and G410 (231 base pairs), as detected by Pertea et al. More precisely G408 (two exons) corresponds to the exons 7 and 8 of FIG. 10A (see alignments at the DNA sequence level in FIG. 10), and G410 corresponds to the exon 9 of FIG. 10A (alignments in FIG. 10C).

The example in FIG. 10D is relative to the region between the genes PFC0780w and PFC0785c (as reported in the original annotation). The analysis of PFC0785c is coherent with the physics. The end of PFC0780w (reported as a simple gene in the database annotation, represented partially in FIG. 10D) is contradicted by the physics. In addition, the physics suggests the existence of a series of coding sequences between the two genes. As represented in FIG. 10E, we can associate 9 exons with the stability analysis, which can be appended to the gene, PFC0780w.

The gene in FIG. 10F (PFC0813c), with 9 exons (coding sequence: 569 base pairs) corresponds to the gene G529 (5 exons, coding sequence: 339 base pairs) in Pertea et al. In this case, only a partial overlap is achieved between the two analyses. It is difficult however to assess the origin of the divergences, as in Pertea et al. no details are provided concerning the analyses (in terms of splicing sites, etc).

Concerning the comparison with other methods, it can be noticed that coding sequences such as those in FIG. 20E (missed in the original annotation) am still missed in the reanalysis of chromosome 3 by Pertea et al. Also, the method used in this reanalysis of chromosome 3 misses a rather large series of potential genes in the chromosome 2 itself, as illustrated by the various examples above. An exhaustive description of such missed genes, as detected by the physics, will be presented elsewhere. It appears that in most cases the missed genes (or coding regions appended to identified genes, as in the example in FIG. 10E) are rather complex ones, with series of small exons. Also, most often, the new genes (predicted on the basis of the physics) do not display any significant similarity with known genes. This observation is not really surprising as extensive similarity searches were performed for the original annotations of the two chromosomes.

3.3.3 Low-Stability Regions within Large Open Reading Frames

In FIG. 13 we consider 5 examples of genes with low-stability regions observed within large open reading frames

The gene in FIG. 13A is predicted with 2 exons (blue) in the database annotation. The physics analysis agrees with the first exon, and indicates the presence of an additional exon at the end of the sequence (f). We observe 3 sharp low-stability regions within the exon 2 of the database annotation. Is it possible to assemble coherently a gene with these low-stability regions treated as introns? The answer is yes and the corresponding hypothetical gene is represented in green (with exons a to f, coordinates in the legend).

In FIG. 13B the database annotation predicts a simple gene (blue). We can observe a low-stability region within the predicted gene, whose right-end frontier is contradicted by the physics. As for the example above, we perform an exon-assembly in which we assimilate the low-stability region to an intron. The result of the procedure is represented as the gene in green (a to c, coordinates in the legend). It can be noticed that the right-end frontier of the exon b is now coherent with the physics. In the analysis we have taken into account an additional exon (c), as suggested by the physics.

The gene in FIG. 13C is also predicted in the database annotation as a simple gene (blue). The physics analysis displays a series of low-stability regions, with one of them of rather large size, as compared to the very sharp peaks in the first example above. In the alternative annotation (in green) we assimilate the low-stability regions to introns, taking into account the proper donor and acceptor sites. In this case, however, a complete exon-assembly is not performed.

In the gene in FIG. 13D we can observe a rather complex landscape for the low-stability regions within the large coding domain. On the other hand, in the case of the gene in FIG. 13E we observe low-stability regions of the ‘attenuated’ type, drawn only by the T60 curve.

With the completion of several large sequencing projects (H. sapiens, D. melanogaster, etc) the proper identification of genes is now the major limiting step in genome projects, most notably for the annotation of complex eukaryotic genomes. Recently, this situation was strikingly illustrated by the controversial reports concerning the number of human genes, as well as communications highlighting the difficulties in the identification of genes in D. melanogaster (Karlin, S., Bergman, A. & Gentles, A J. Nature 411, 259-260 (2001)) or P. falciparum (Pertca. M., Salzberg, S. L. & Gardner, M. J. Nature 404, 34 (2000); Lawson, D., Bowman, S. & al., Nature 404, 34-35 (2000)). We report here the experimental validation of a new physics-based gene-identification (‘PBGI’) method, illustrated for the P. falciparum genome.

Most often, the identification of genes relies on homology information. When such information is not available the ab initio, identification of genes can be a hazardous process, relying on various elaborate pattern-recognition models (involving frequently training steps, etc). In the PBGI scheme, the ab initio identification of genes is based on the—thermal—stability properties of the DNA double-helix (Yerarmian, E. Gene 255,139-150 (2000); Yeramian, E. Gene 255, 151-168 (2000)), as described by the classical helix-coil transition model (Poland, D. & Scheraga, H. R. Theory of Helix Coil Transitions in Biopolymers. (Academic Press, New York, 19-10); Yeramian, E., Schaeffer, F., Caudron, B., Clayerie, P. & Buc, H. Biopolymers 30, 481-497 (1990)). With appropriate algorithmic formulations (Yeramian, E., Schaeffer, F., Caudron, B., Clayerie, P. & Buc, H. Biopolymers 30, 481-497 (1990)), it is possible to implement such a structural model at the level of large genomic sequences. The study of cloned genes demonstrated that in P. falciparum it is possible to discriminate, in almost every case,. the coding and non-coding regions on the basis of the DNA stability properties (Yeramian, E. Gene 255, 151-168 (2000)). In this discrimination, coding regions, simple genes, or exons in split genes, are precisely delimited as high-stability regions. The correspondence holds for coding regions whose lengths extend, roughly, from 10 base pairs up to 10,000 base pairs. For the complete gene analysis, the information provided by the physics is combined with classical sequence analyses (e.g. open reading frames, identification of proper splice sites at the boundaries). Such a combination also allows the resolution of ambiguities when, for example, a single stability region harbours two distinct exons (Yeramian, E. Gene 255, 151-168 (2000).

The aim of this work is largely practical: testing the idea that the intrinsic structural properties of DNA may be used for the identification of genes, and the annotation of genomes. For the P. falciparum genome it appears that, indeed, an accurate and versatile gene-prediction scheme can be elaborated based on the stability properties of the DNA double-helix. In contrast to other predictive methods, genes are identified immediately by mere visual inspection of the stability curves. The detailed landscapes associated with the stability curves provide in a straightforward manner the basic information concerning the exon-intron boundaries. With the proper calibration of the predictive criteria, automatic procedures may be constructed to detect stability frontiers and perform exon-assembly in a self-consistent fashion. The treatments here demonstrate that, in order to be meaningful, automatic procedures must rely on a series of ‘knowledge rules’, elaborated from the detailed study of cloned genes. The proper detection of exons in the EHSR domain, as defined above, represents one example for the importance of such rules. In any case, the structural analyses clearly provide a useful complement to other predictive methods, for the study of P. falciparum sequences.

Beyond the analyses of P. falciparum, the possible extension of the predictive scheme to other genomes, and organisms must be explored in detail. Perhaps this gene-identification procedure may not be equally effective for all genomes. It seems however unlikely that P. falciparum represents an isolated ‘accident’ within the various genomes. Beyond the practical purposes, such explorations should open in any case interesting perspectives to understand the role of structural effects in the evolutionary shaping of genomes. Several such questions are raised by the results here, on the evolutionary implications for the structural description of split genes. Whereas the correspondence between physics and genetics at the level of simple genes (see above) could be interpreted in rather simple—speculative—schemes, it is more difficult to imagine the origin of the correspondence at the level of split genes. We may even wonder why such a relation should exist at all, as splicing events occur at the RNA level and not at the DNA level. Possible answers to such questions will have to await more global overviews of the physics of split genes, throughout genomes and organisms.

For the present time, limiting ourselves to the results obtained with P. falciparum it is interesting to point out two links between the practical considerations (the aim in this paper) and the evolutionary perspectives (as discussed above), concerning split genes.

The first link concerns the low-stability regions observed within certain large open reading frames, discussed throughout this paper. Only detailed experimental studies will allow to decide whether such regions represent artefacts, in the predictive scheme. An example such as the one above concerning different stage expressions could hint that functional effects could be attached to such regions, at least in certain cases. If the low-stability regions do indeed correspond to artefacts, they may be easily dismissed from the analyses (based on ORF informations). Even in such a case, the low-stability regions—which we could call for the moment ‘ghost introns’—should be very interesting to study from the evolutionary point of view. Analyses such as those performed above demonstrate that—at least in the considered cases a complete split-gene structure can be associated with ghost-introns, relative to the classical sequence-analysis criteria (proper splice signals and ORF-based exon-assembly). It is then legitimate to ask whether ghost-introns might represent vestiges of ancient introns? The second link concerns the description of EHSR regions (as defined above). The proper handling of such regions is fundamental for the practical purposes. In evolutionary terms it is interesting to note that the definition of such regions could be cast straightforwardly in the 'stability assimilation, concept, as developed in the companion paper. In the cuvette configuration, the description of an intron not distinguishable from the two adjacent exons (in terms of stability) is strictly comparable to the ‘erasure’ of certain intergenes as seen in the S. cerevisiae genome (see above). As for the shoulder configuration, it can be also described in terms of stability homogenisation (the stability of an exon becoming non-distinguishable from that of the adjacent intron). Such observations, at the DNA structural level, raise questions about the possible involvement of erasure and homogenization mechanisms common to the. different kinds of coding regions (simple genes as well as exons). Very little is presently known about the evolutionary histories leading from simple genes to split genes, or vice versa. The accumulation of structural informations may help to bridge such gaps. Plasmodium falciparum seems likely to represent an important chapter in the structural reading of the genome evolution book.

The sequence of the complete genome of P. falciparum will become available soon. The experimental validation of the PBGI scheme as a predictive tool becomes then all the more important, as for the two already published chromosomes (chromosome 2 by the TIGR (Gardner, M. J. & al. Science 282, 1126-1132 (1998) and chromosome 3 by the Sanger (Bowman, S. & al. Nature 400, 532-538 (1999)) significant differences can be observed between the PBGI analyses and the official annotations. Also, the two annotation methods themselves (‘method2’—GimmerM program—for chromosome 2, and ‘method3’ for chromosome 3) can lead to different results (notably following the reanalysis of chromosome 3 by method2 (Pertca. M., Salzberg, S. L. & Gardner, M. J. Nature 404, 34 (2000)). In the general perspective of difficulties associated with gene identification, it is therefore interesting to present the conclusions of the experimental testing of the PBGI predictions in the light of such cross-analyses. As reported in (Pertca. M., Salzberg, S. L. & Gardner, M. J. Nature 404, 34 (2000), the original analysis of chromosome 3 by method3 missed a series of genes (25 genes following (Pertca. M., Salzberg, S. L. & Gardner, M. J. Nature 404, 34 (2000)). However, following the PBGI analyses, the analysis of chromosome 2 by method2 missed a comparable number of genes (30). Most missed genes are complex ones, with a series of small exons. Indeed the identification of small exons can be difficult. This results not only in the failure to identify complex, multi-exon, genes, but also in the missing of small additional exons belonging to identified genes.

For the experimental testing of the above conclusions, 20 illustrative examples were selected from the predictions. The inventor took a well characterised cDNA library as our source of mRNA (Rawlings, D. J. & Kaslow, D. C., J. Biol. Chem. 267, 3976-3982 (1992), and performed PCR on both genomic DNA and cloned cDNA. As expected, the cDNA library did not contain transcripts for all examples chosen, but we were able amplify 14 out of the 20 predictions. Complete sequencing was performed on 11 examples, including 3 cases for which the sizes of the transcripts did not agree with the predictions (as a self-coherency test for the accuracy of the predictions). In the latter 3 cases, where the size was discordant, the sequence revealed that the transcripts were not specific for the genes analysed. In the 8 other cases the sequencing confirmed the predictions.

The various conclusions above are summarized by the FIG. 14a, for a genomic region of chromosome 2. Various coding regions correspond to high-stability domains (helical state), as sharply delimited in the probability curves for the opening of the double-helix (5 different temperatures, used throughout all the analyses of P. falciparum). The simple gene in red illustrates the agreement between the physics and the original annotation. Following the physics, a small exon must be added to the gene in green (original annotation as a simple gene). The gene in blue (4 exons) corresponds to a PBGI prediction confirmed by sequencing (and missing in the annotation). All the examples in FIGS. 14b to 14f correspond to PBGI predictions confirmed by sequencing (all examples from chromosome 2, with the exception of FIG. 14e from chromosome 3). FIG. 14b corresponds to a relatively simple missed gene (2 exons). FIGS. 14c and 14d correspond to more complex missed genes. The missed gene in FIG. 14c (6 exons) gives an insight onto the accuracy of the PBGI predictions (with an indication of the discrepancies between the prediction of this gene, as reported in (Yeramian, E., Gene, 255, 151-168 (2000), and the experimental results, see legend. For the complex missed gene in FIG. 14d (13 exons), only the first 5 exons were tested, and confirmed, experimentally. In this example, the physics alone did not detect the intron between exons 1 and 2. However, simple ORF (open reading frame) analysis predicted the presence of the intron in the stability region harbouring exons 1 and 2. A similar situation can be observed for exons 8 and 9 (FIG. 14d). The examples in FIGS. 14e and 14f illustrate cases of missed exons for otherwise identified genes (2 exons for the gene in FIG. 14e, in chromosome 2, and 9 exons for the gene in FIG. 14f, in chromosome 3), Interestingly, the missing 9 exons in FIG. 14f were missed in the original annotation and in the cross-annotation mentioned above (Pertca, M., Salzberg, S. L. & Gardner, M. J., Nature 404, 34 (2000). This figure is also illustrative for the accuracy of the PBGI predictions, with the indication of the discrepancies between the original predictions (in Yeramian, E., Gene, 255, 151-168 (2000)) and the experimental results, (see legend).

Clearly gene identification is a difficult problem and various successful methods should be considered in conjunction for increasing the accuracy and reliability of the analyses and predictions. The experimental proof provided here argues that PBGI is an additional powerful tool for the annotation of the P. falciparum genome. In contrast to specialized approaches (with training methods and sets) the properties at the basis of PBGI are ‘universal’ (with only the temperatures used as ‘probes’ varying, following the gross GC contents of the genomes). Preliminary studies indicate that PBGI will be helpful in the identification of genes in a series eukaryotic genomes (P. vivax, D. melanogatler, A. thaliana, H. Sapiens, etc).

Identification of Genes in Eukaryotic Genomes, in General:

Various aspects of gene identification as described for Plasmodium falciparum are generalized and applied to various eukaryotic genomes.

As such, the physics-based gene identification procedure as described here can be used for the identification and the discovery of new genes in various eukaryotic genomes (Homo sapiens, Drosophila melanogaster, Anopheles gambiae, etc).

In order to illustrate this point, we consider below a given gene of the large subunit of RNA polymerase II (in short pol-gene), and consider its physics-based analysis in several genomes. In FIG. 15, the corresponding results are presented for C. elegans (12 exons presented in blue: Accession Number U53333), D. melanogaster (4 exons in blue: Accession Number M2743 1), T. arabidopsis (a large number of exons, clustered at the right side of the presented sequence), and P. falciparum (a simple gene, in blue).

Following such analyses, it appears that the physics-based gene identification method is not necessarily very accurate in the detection of the various exons in a gene, such as the one above for T. arabidopsis, when the stability regions corresponding to the different exons are not sharply distinguished from each other. On the other hand, the physics-based method is very powerful in the straightforward identification of genes for which the exons are sharply discriminated from each other, such as for the examples above in C. elegans and D. melanogaster.

The above result can then be used for the practical discovery of potential new genes in a genome such as Homo sapiens. As an illustration, we consider below a genomic sequence from H. sapiens (Accession AP001754). In the original annotation, genes are reported only for the first half of the genomic sequence (length 340,000 bp), and these genes are represented in red and blue. See FIG. 16.

With the physics-based gene identification scheme, potential new genes are discovered in this second half, as represented partially (exons in red, in the region 270 to 300 kbp, and further zooming as shown in FIG. 17). Part of the above analysis is presented in FIG. 21 in more detail. In the original genomic sequence the coding regions are in green, regions as discovered by the physics-based are highlighted in blue text (the non-coding regions are in green, and the splice sites in magenta).

Eukaryotic Genomes, Particular Case of Anopheles gambiae:

For the gene identification procedure as described above, it appears that the genomes of insects represent particularly favorable cases, with possibly the identification of all genes (and almost all exons). This situation is illustrated with D. melanogaster and A. gambiae, for example.

In the case of A. gambiae, notably, with the conditions adopted throughout this document, it appears that very few temperatures (74° C., 75° C., and 76° C. with the conditions chosen, and described above) could be enough as a matter of fact for the proper detection of genes and exons. This feature is illustrated below with the gene AgProPO (Accession Number: AF031626).

All the known exons are detected by the physics-based gene identification method. An interesting feature is that the small (or very small) variations in the curves corresponding to temperatures T=75° C. and T=76° C can allow to detect very precisely the presence of introns (such as between the last two exons, at the end of the sequence).

Identification of Genes which are Transcribed but not Translated:

The importance of genes which are transcribed but not translated is becoming increasingly clear. The corresponding RNAs can be involved in a series of fundamental genetic regulations. The physics-based gene identification procedure as described here appears to allow the detection of such genes, in the same way than for ‘ordinary’ genes. The point is illustrated here with genes from Plasmodium falciparum.

We consider as an example the gene described in the following reference: “An EBA175 homologue which is transcribed but not translated in erythrocytic stages of Plasmodium. falciparum” (Triglia, T., Thompson, J. K. and Cowman, A. F. Mol. Biochem. Parasitol. 116 (1), 55-63 (200 1)). The physics-based gene analysis as displayed in FIG. 19 (with the usual parameters for Plasmodium analyses) shows that the exons of the non-translated gene (join(1 . . . 4021, 4191 . . . 4269, 4410 . . . 4543, 4637 . . . 4698)) are detected in the same way than for ‘usual’ genes.

Identification of Multi-Functional (Bi-Functional, etc) Genes:

In the genes identified by the physics-based method described here, there are instances in which ‘ghost introns’ or ‘ghost intergenes’ are observed: stability domains which appear as introns or intergenes, but as a matter of fact are part of the considered gene. As demonstrated by a series of known cases, it appears that such ‘spurious’ stability domains, in the gene identification procedure, can lead to the identification of multi-functional genes (with the different ‘functional’ domains appearing as separate stability domains, as for independent genes). As such the physics-based gene identification procedure as described here can be a useful tool for the detection and identification of such multifunctional genes,

As an illustrative case we consider the G6PD gene in Plasmodium falciparum (Accession Number X74988) whose physics analysis is displayed in FIG. 20 (following the usual parameters used throughout this document). The stability domain (353-1234) within this gene appears as an “intergene”. As desribed in the reference “Glucose-6-phosphate dehydrogenase-6-phosphogluconolactonase. A novel bifunctional enzyme in malaria parasites” (Clarke J L, Scopes D A, Sodeinde O, Mason P J; Eur J. Biochem 2001 Apr, 268(7):2013-2019), it appears that indeed the two stability domains thus displayed in the physics (between 184-353 and 1234-2916) correspond to the two functional domains of the bi-functional gene G6PD.

This result can be generalized to other organisms and genomes.

### REFERENCES

Bloomfield, V. A., Cyothers, D. M., Tinonco, I. Jr. 1974. Physical Chemistry of Nucleic Acids. Harper and Row, New York.

Cantor, R. C., Schimmel, P. R., 1980. Biophysical Chemistry. Part M: the behaviour of biological macromolecules. W. H. Freeman and company, New York.

Coissac, E., Maillier, E., Netter, P., 1997. A comparative study of duplications in bacteria and eukaryotes: the importance of telomeres. Mol. Biol. Evol. 14, 1062-1074.

Cole, S. T. et al., 1998. Deciphering the biology of Mycobacierium tuberculosis from the complete genome sequence. Nature 393,537-544.

Fixman, M., Freire, J. J., 1977. Theory of DNA melting curves. Biopolymers 16, 2693-2704.

Gillespie, J. H., 1991. The causes of molecular evolution. Oxford University Press, New York, Oxford.

Goffeau, A. et al., 1997. The Yeast Genome Directory. Nature 387 (Suppl.), 1-105.

Gotoh, O., Tagashira, Y., 1981 a. Stabilities of nearest-neighbour doublets in double-helical DNA determined by fitting calculated melting profiles to observed profiles. Biopolymers 20, 1033-1042.

Gotoh, O., Tagashira, Y., 1981b. Locations of frequently opening regions on natural DNAs and their relation to functional loci. Biopolymers 20, 1043-1058.

Gotoh, O., 1983. Prediction of melting profides and local helix stability for sequenced DNA. Adv. Biophys, 16, 1-52.

Grosberg, A. Y., Khokhlov, A. R., 1994. Statistical physics of macromolecules. AIP Press, New York, 302-322.

Jacobson, H., Stockmayer, W. H., 1950. Intermolecular reactions in polycondensation. 1. The theory of linear systems. J. Chem. Phys. 18, 1600-1606.

King, J. K., 1993. Stability, structure and complexity of yeast chromosome III. Nucleic Acids Res. 21, 4239-4245.

Lalo, D., Stettle, S., Mariotte, S., Slonimski, P. P., Thuriaux, P., 1993. Two yeast chromosomes are related by a fossil duplication of their centromeric regions. C. R. Acad. Sci. III 316, 367-373.

Li, W. H., 1997. Molecular Evolution. Sinauer Associated, Sunderland Massachusetts, 353-354.

Lyubchenko, Y. L., Frank-Kamenetskii, M. D., Vologodskii, A. V., Lazurkin, Y. S. Gause, G. G., 1976. Fine structure of DNA melting curves. Biopolymers 15, 1019-1036.

Lyubcheako, Y. L., Vologodskii, A. V., Frank-Kamenetskii, M. D., 1978. Direct comparison of theoretical and experimental melting profiles for RFII ΦX174 DNA. Nature 271, 28-31.

Osawa, S. et al., 1987. Cold Spring Harbor Symp. Quant. Biol. 70, 777-789.

Poland, D., Scheraga, H. R., 1970. Theory of Helix Coil Transitions in Biopolymers. Academic Press, New York.

Schaeffer, F., Yeramidan, E., Lilley, D. M J., 1989. Long-range structural effects in supercoiled DNA: statistical thermodynamics reveals a correlation between calculated cooperative melting and contextual influence on cruciform extrusion. Biopolymers 28, 1449-1473.

Steger, G., 1994. Thermal denaturation of double-stranded nucleic acids: prediction of temperatures critical for gradient gel electrophoresis and polymerase chain reaction. Nucleic Acids Res. 22, 2760-2768.

Suyama, A., Wada, A., 1983. Correlation between thermal stability maps and genetic maps of double-stranded DNAs. J Theoret. Biol. 105, 133-145.

Yeramian, E., Clayerie, P., 1987. Analysis of muldexponential functions without a hypothesis as to the number of components. Nature 326, 169-174.

Yeramian, E., Schaeffer, F., Caudron, B., Clayerie, P., Buc, H., 1990. An optimal formulation of the matrix method in statistical mechanics of one-dimensional interacting units: efficient iterative algorithmic procedures. Biopolymers 30,481-497.

Yeramian, E., 1994. Complexity and tractability. Statistical mechanics of helix-coil transitions in circular DNA as a model problem. Europhys. Lett. 25, 49-55.

Yeramian, E., 2000. The physics of DNA and the annotation of the Plasmodium falciparum genome. Gene 255, 139-150.

Wada, A., Yabuki, S., Husimi, Y., 1980. Fine structure in the thermal denaturation of DNA: high-resoiution spectrophotometric studies. CRC Crit. Rev. Biochem. 9, 87-144.

Wartell, R. M., Benight, A. S., 1985. Thermal denaturation of DNA molecules: a comparison of theory with experiment. Physics Reports 126, 67-107.

Watson, J. D., Crick, F. H. C., 1953. Molecular structure of nucleic acids. A structure for Deoxyribose Nucleic Acid. Nature 171, 737-738.

Wells, R. D. eL al., 1980. DNA structure and gene regulation. Prog. Nucl. Acid Res. Mol. Biol. 24, 167-267.

Wolfe, K. H. Shields, D. C., 1997. Molecular evidence for an ancient duplication of the entire yeast genome. Nature 387, 708-713.

Wolff, G., Plante, I., Lang, B. F., Kueck, U., Burger, G., 1994. Complete sequence of the mitochondrial DNA of the chlorophyte alga Prototheca wicckerhamii. Gene content and genome organization. I Mol. Biol. 237,75-86.

Baldi, P., Brunak, S., 1998. Bioinformatics. The machine learning approach. MIT Press, Cambridge, Mass.

Bhaduri-Mclntosh, S., Vaidya, A. B., 1996. Molecular characterization of a Plasmodium falciparum gene encoding the mitochondrial phosphate carrier. Mol. Biochem. Pasasitol. 78, 297-301.

Bowman, S. et al., 1999. The complete nucleotide sequence of chromosome 3 of Plasmodium falciparum. Nature 400, 532-538.

Crothers, D. M., Kallenbach, N. R., 1966. On the helix-coil transition in heterogeneous polymers. J. Chem. Phys. 45, 917-927.

Fox, B. A., Bzik, D. J., 1994. Analysis of stage-specific transcripts of the Plasmodium falciparum serine repeat antigen (SERA) gene and transcription from the SERA locus. Mol. Biochem. Parasitol. 68, 133-144.

Gardner, M. J., et al., 1998. Chromosome 2 sequence of the human malaria parasite Plasmodium falciparum. Science 282, 1126-1132.

Holloway, S. P., Gerousis, M., Delves, C. J., Sims, P. F. G., Scaife, J. G., Hyde, J. E. 1990. The tubulin genes of the human malaria parasite Plasmodium falciparum: Their chromosomal location and sequence analysis of the alpa-tubulin II gene. Mol. Biochem. Parasitol. 43, 257-270.

Kimura, M., Yamaguchi, Y., Takada, S., Tanabe, K., 1993. Cloning of a Ca(2+)-ATPase gene of Plasmodium falciparum and comparison with vertebrate Ca(2+)-ATPases. J. Cell. Sci. 104, 1129-1136.

Knapp, B., Nau, U., Hundt, E., Kuepper, H. A., 1991. Demonstration of alternative splicing of pre-mRNA expressed in the blood stage form of Plasmodium falciparum. J. Biol. Chem. 266, 7148-7154.

Kun, J. F., Hibbs, A. R., Saul, A., McColl, D. J., Coppel, R. L., Anders, R. F., 1997. A putative Plasmodium falciparum exported serine/threonine protein kinase. Mol. Biochem. Parasitol. 85, 41-51.

Lawson, D., Bowman, S., Barrell, B., 2000. Bioinformatics: Finding genes in Plasmodium falciparum. Nature 404, 34-35.

Li, W. B., Bzik, D. J., Tanaka, M., Gu, H., Fox, B. A., Inselburg, J., 1991. Characterization of the gene encoding the largest subunit of Plasmodium falciparum RNA polymerase III. Mol. Biochem. Parasitol. 46, 229-240.

Luersen, K, Walter, R. D., Muller, S., 1999. The putative gamma-glutamylcysteine synthetase from Plasmodium falciparum contains large insertions and a variable tandem repeat. Mol. Biochem. Parasitol. 98, 131-142.

Marck, C., 1988. ‘DNA Strider’: a ‘C’ program for the fast analysis of DNA and protein sequences on the Apple Macintosh family of computers. Nucleic Acids Res 16, 1829-1836

Marshall, V. M., Coppel, R. L., 1997. Characterisation of the gene encoding adenylosuccinate lyase of Plasmodium falciparum. Mol. Biochem. Parasitol. 88 (1-2), 237-241.

Marshall, V. M., Tiequao, W., Coppel, R. L., 1998. Close linkage of three merozoite surface protein genes on chromosome 2 of Plasmodium falciparum. Mol. Biochem. Parasitol. 94, 13-25.

Mohrle, J. J., Zhao, Y., Wemli, B., Franklin, R. M., Kappes, B., 1997. Molecular cloning, characterization and localization of PfPK4, an eIF-2alpha kinase-related enzyme from the malarial parasite Plasmodium falciparum. Biochem. J. 328 (Pt 2), 677-687.

Pertea, M., Gardner, M. J., Salzberg, S. L., 2000. Bioinformatics: Finding genes in Plasmodium falciparum. Nature 404, 34.

Poland, D., Scheraga, H. R., 1970. Theory of Helix Coil Transitions in Biopolymers. Academic Press, New York.

Prasartkaew, S., Zijlstra, N. M., Wilairat, P., Overdulve, J. P., de Vries, E., 1996. Molecular cloning of a Plasmodium falciparum gene interrupted by 15 introns encoding a functional primase 53 kDa subunit as demonstrated by expression in a baculovirus system. Nucleic Acids, Res. 24, 3934-3941.

Reeder, J. C., Cowman, A. F., Davem, K. M., Beeson, J. G., Thompson, J. K., Rogerson, S. J., Brown, G. V., 1999. The adhesion of Plasmodium falciparum-infected erythrocytes to chondroitin sulfate A is mediated by PFEMP1. Proc. Natl. Acad. Sci. U.S.A. 96, 5198-5202.

Ross-Macdonald, P. B., Graeser, R., Kappes, B., Franklin R., Williamson, D. H., 1994. Isolation and expression of a gene specifying a cdc2-like protein kinase from the human malaria parasite Plasmodium falciparum. Eur. J. Biochem. 220, 693-701.

Salzberg, S. L., Delcher, A. L., Kasif, S., White, O., 1998. Microbial gene identification using interpolated Markov models. Nucleic Acids Res. 26, 544-548.

Stafford, W. H., Stockley, R. W., Ludbrook, S. B., Holder, A. A., 1996. Isolation, expression and characterization of the gene for an ADP-ribosylation factor from the human malaria parasite, Plasmodium falciparum. Eur. J. Biochem. 242, 104-113.

Triglia, T., Cowman, A. F., 1999. Plasmodium falciparum: A Homologue of p-Aminobenzoic Acid Synthetase. Exp. Parasitol. 92, 154-158.

Trottein, F., Triglia, T., Cowman, A. F., 1995. Molecular cloning of a gene from Plasmodium falciparum that codes for a protein sharing motifs found in adhesive molecules from mammals and plasmodia. Mol. Biochem. Parasitol. 74, 129-141.

Williamson, K. A., Criscio, M., Kaslow, D. C., 1993. Cloning and expression of the gene for Plasmodium falciparum transmission-blocking target antigen, Pfs230. Mol. Biochem. Parasitol. 58, 355-358.

Wootton, J. C., Federhen, S., 1993. Statistics of local complexity in amino acid sequences and sequence databases. Computers & Chemistry 17, 149-163.

Zhao, Y., Kappes, B., Franklin, R. M., 1993. Gene structure and expression of an unusual protein kinase form Plasmodium falciparum homologous at its carboxyl terminus with the EF hand calcium-binding. J. Biol. Chem. 268, 4347-4354.

What is claimed is:

1. Method for the identification of genes and genetic signals based on the structural properties of DNA double-helix comprising the following steps:
A—using the classical physical model of helix-coil transitions,
B—calculating stability curves (probabilities of opening of the DNA double-helix, along a given sequence) by algorithmic methods,
C—determining the disruption in the linear DNA for different temperatures,
D—analysing the stability curves for the detection of genetic signals (disruption of the double-helix) or the identification of coding regions (simple genes or exons in split genes, regions of high thermal stability), and optionally,
E—based on the structural informations, performing classical sequence analysis (donor/acceptor sites, start and codon stops, in correspondence with the frontiers identified in the stability curves and open reading frames analyses) for completing the identification of genes.

2. Method according to claim 1, characterized by an identification and ab initio prediction method of coding regions comprising simple genes (without introns) and/or exons in split genes (containing exons) in various genomes.

3. Method according to claim 1 and claim 2 characterized by a procedure for the annotation of the various genomes comprising simple genes (without introns) and/or exons in split genes (containing introns) in various genomes characterized by performing the steps A to E of claim 1.

4. Method according to claim 1 characterized by an ab initio prediction method for the identification of genetic signals in various genomes comprising promoters or regulatory sequences characterized by the propensity of opening the DNA double helix [easily melt-region]

5. Use of the method as claimed in claims 1 to 4 for the identification of genes and genetic signals in various genomes

6. Use of the method according to claim 5 wherein the genome is an eukaryotic genome.
